SEQUENCE LISTING (12 sequences)

SEQ ID NO: 1 - protein, 5 aa, Artificial Sequence
  source: /note=“Description of Artificial Sequence Synthetic peptide”
GGGGS

SEQ ID NO: 2 - protein, 110 aa, Artificial Sequence
  source: /note=“Description of Artificial Sequence Synthetic polypeptide”
ARVDQTPQTITKETGESLTINCVLRDSNCALSSTYWYRKKSGSTNEENISKGGRYVETVNSGSKSFSLRINDLTVEDSGTYRCNVVQYPQYPNYFWCDVYGDGTAVTVNA

SEQ ID NO: 3 - protein, 109 aa, Artificial Sequence
  source: /note=“Description of Artificial Sequence Synthetic polypeptide”
ARVDQTPQTITKETGESLTINCVLRDSNCALSSTYWYRKKSGSTNEENISKGGRYVETVNSGSKSFSLRINDLTVEDSGTYRCNVVQYPSYNNYFWCDVYGDGTAVTVN

SEQ ID NO: 4 - protein, 111 aa, Artificial Sequence
  source: /note=“Description of Artificial Sequence Synthetic polypeptide”
ARVDQTPQTITKETGESLTINCVLRDSNCELSSTYWYRKKSGSTNEESISKGGRYVETVNSGSKSFSLRINDLVVEDSGTYRCNVQQFPSSSNGRYWCDVYGGGTAVTVNA

SEQ ID NO: 5 - protein, 114 aa, Artificial Sequence
  source: /note=“Description of Artificial Sequence Synthetic polypeptide”
ARVDQTPQTITKETGESLTINCVLRDSNCALPSTYWYRKKSGSTNEESISKGGRYVETVNSGSKSFSLRINDLTVEDSGTYRCKVIAQLSSILRGCNYRKHDVYGDGTAVTVNA

SEQ ID NO: 6 - protein, 107 aa, Artificial Sequence
  source: /note=“Description of Artificial Sequence Synthetic polypeptide”
ARVDQTPQTITKETGESLTINCVLRDSNCALSSTYWYRKKSGSTNEENISKGGRYVETVNSGSKSFSLKINDLTVEDSGTYRCNVVGTWCMSWRDVYGGGTAVTVNA

SEQ ID NO: 7 - protein, 112 aa, Artificial Sequence
  source: /note=“Description of Artificial Sequence Synthetic polypeptide”
ARVDQTPQTITKEEGESLTINCVLRVHGRALASTSWYRKKSGSTREETISKGGRYVETVNSGSKSFSLRINDLTVEDSGTYRCNVYGLSFGDIEGVKKIDVYGDGTAVTVNA

SEQ ID NO: 8 - protein, 15 aa, Artificial Sequence
  source: /note=“Description of Artificial Sequence Synthetic peptide”
GGGGSGGGGSGGGGS

SEQ ID NO: 9 - protein, 214 aa, Artificial Sequence
  source: /note=“Description of Artificial Sequence Synthetic polypeptide”
AIVLIQSPATLSVTPGDSVSLSCRASQTISNNLHWYQQKSHESPRLLIKSASLAISGIPSRFSGSGSGTDFTLSISSVETEDFGMYFCQQSNSWPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, Artificial Sequence
  source: /note=“Description of Artificial Sequence Synthetic polypeptide”
EVQLQQSGPELVKPGASMKISCKTSGYSFTAYFMNWVKQSHGKSLEWIGRINPNNGDTFYTQKFKGKATLTVDKSSNTAHMELLSLTSEDSAIYYCGRRDYFGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 573 aa, Artificial Sequence
  source: /note=“Description of Artificial Sequence Synthetic polypeptide”
ARVDQTPQTITKETGESLTINCVLRDSNCALSSTYWYRKKSGSTNEENISKGGRYVETVNSGSKSFSLRINDLTVEDSGTYRCNVVQYPQYPNYFWCDVYGDGTAVTVNAGGGGSGGGGSGGGGSEVQLQQSGPELVKPGASMKISCKTSGYSFTAYFMNWVKQSHGKSLEWIGRINPNNGDTFYTQKFKGKATLTVDKSSNTAHMELLSLTSEDSAIYYCGRRDYFGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 578 aa, Artificial Sequence
  source: /note=“Description of Artificial Sequence Synthetic polypeptide”
EVQLQQSGPELVKPGASMKISCKTSGYSFTAYFMNWVKQSHGKSLEWIGRINPNNGDTFYTQKFKGKATLTVDKSSNTAHMELLSLTSEDSAIYYCGRRDYFGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSARVDQTPQTITKETGESLTINCVLRDSNCALSSTYWYRKKSGSTNEENISKGGRYVETVNSGSKSFSLRINDLTVEDSGTYRCNVVQYPQYPNYFWCDVYGDGTAVTVNAGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0000998 A1
(43) Pub. Date: Jan. 5, 2023

(54) BBB-SHUTTLING-VNARS CONJUGATED TO NEUROTROPHIC AGONIST ANTIBODIES TO TREAT NEURODEGENERATIVE DISEASES AND CONDITIONS

(71) Applicant: OSSIANIX, INC., Philadelphia, PA (US)

(72) Inventors: Julia Lynn Rutkowski, Bryn Mawr, PA (US); Frank Walsh, Bryn Mawr, PA (US); Elizabeth H. Sinclair, Zurich (CH); Pawel Stocki, Hertfordshire (GB)

(73) Assignee: OSSIANIX, INC., Philadelphia, PA (US)

(21) Appl. No.: 17/778,665

(22) PCT Filed: Nov. 20, 2020
(86) PCT No.: PCT/US2020/061533

§ 371 Date: May 20, 2022

Related U.S. Application Data
(60) Provisional application No. 62/939,522, filed on Nov. 22, 2019.

(51) Int. Cl. A61K 47/64; A61K 47/68; A61P 25/16; C12N 15/63

(52) U.S. Cl. CPC A61K 47/6425; A61K 47/68; A61P 25/16; C12N 15/63

(57) ABSTRACT

The present disclosure relates to conjugates for delivering therapeutics across the blood brain barrier (BBB) and more particularly to conjugates comprising at least one BBB-shuttling VNAR domain operably linked to a neurotrophic agonist antibody (NAAb), with the conjugate being capable of uptake across a mammalian blood brain barrier (BBB) in a therapeutically-effective amount. These conjugates are useful for treating neurodegenerative diseases, conditions which responds to activation of a neurotrophin receptor as well as for stimulating neuronal survival, growth, repair or regeneration.

### CROSS REFERENCE TO RELATED APPLICATION

This PCT application claims the benefit of provisional application U.S. Ser. No. 62/939,522, filed on Nov. 22, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 19, 2020, is named OSX1904-WO1_SL.txt and is 23,637 bytes in size.

### FIELD OF THE INVENTION

The present invention relates to conjugates for delivering therapeutics across the blood brain barrier (BBB) and more particularly to conjugates comprising at least one BBB-shuttling VNAR domain operably linked to a neurotrophic agonist antibody (NAAb), with the conjugate being capable of uptake across a mammalian blood brain barrier (BBB) in a therapeutically-effective amount. These conjugates are useful for treating neurodegenerative diseases, conditions which responds to activation of a neurotrophin receptor, and for stimulating neuronal survival, growth, repair or regeneration.

### BACKGROUND OF THE INVENTION

The interaction of neurotrophins (NGF, BDNF, NT3 and NT4) with their cognate Trk receptors (TrkA, TrkB and TrkC) protects neurons from naturally occurring cell death during development. The neurotrophins' ability to nurture developing neurons spawned numerous studies to determine if they can promote the survival of adult neurons, particularly in the context of neurodegenerative disease or acute brain injury. In this context promising results have been found with BDNF, which by activating the TrkB receptor, has been shown to protect neurons from death in preclinical models of Parkinson's disease and in ischemic lesions. In addition, BDNF can promote functional recovery of injured neurons following spinal cord injury and stimulate the production of new neurons in the adult brain. Loss of BDNF has also been suggested as a contributory factor to the progression of Alzheimer's disease and Huntington's disease, as well as conditions such as depression.

However, the therapeutic potential of BDNF in neurodegenerative diseases, acute brain injury and other neurological conditions has not been realized in the clinical setting, in part due to its relatively short in vivo half-life and to its exclusion from the brain parenchyma following systemic administration. While the identification of agonist antibodies that directly bind the TrkB receptor and mimic the neurotrophic activity of BDNF provides a pathway for the development of reagents with a long in vivo half-life, the challenge of poor blood-brain barrier (BBB) penetration remains, making the systemic delivery of TrkB agonist antibodies limited for treatment of peripheral disorders such as obesity and peripheral neuropathy. Nonetheless, when delivered directly across the BBB by intracerebroventricular (icy) injection prior to an ischemic injury, the TrkB agonist antibody 29D7 enhanced neuronal survival and promoted functional recovery (U.S. Pat. No. 7,750,122).

Moreover, numerous TrkB-selective agonist antibodies and small molecule agonists have been described and characterized for effects in signaling pathways and downstream, in neuronal survival, repair and regeneration (reviewed in Josephy-Hernandez 2017; see also, e.g., Qian 2006; Hu 2010; Xu 2010; Bai 2010; Sahenk 2010; Fouad 2010; Cazorla 2011; Vugmeyster 2013; Perreault 2013; Todd 2014; Kim 2014; Rosenthal 2014; and Merkouris 2018).

For example, the TrkB-selective monoclonal antibody agonist 29D7 has been shown to enhance neuronal survival after optic nerve injury (Hu 2010), to promote neuronal plasticity following cervical spinal cord injury (Fouad 2010) and to increase neuronal survival and behavioral recovery following neonatal hypoxic-ischemic brain injury (Kim 2014). More recently, four TrkB-selective monoclonal antibodies (M3-M6) that activate Trk receptors (i.e., act as antibody agonists) have been generated, and one (M3) shown to have beneficial effects on neuronal survival, neurite extension and synapse restoration (Szobota 2019). Another TrkB agonistic antibody (Ab4B19) has been reported to provide therapeutic benefits in ischemic brain injury models (Han 2019).

Nonetheless, delivery issues persist so considerable interest exists in “high-jacking” receptor-mediated transcytosis pathways found on the BBB endothelial cells to carry therapeutic antibodies or enzymes from the periphery to the brain parenchyma, with the transferrin receptor 1 (TfR-1) being the most widely studied of such receptors (Johnsen 2019). However, for a variety of reasons, conventional antibodies have not realized this potential.

Single domain antibodies that occur naturally in the shark are particularly attractive for the development of next generation biotherapeutics, and particularly to act as trojan horses for delivering (bio)therapeutics across the BBB and into the brain. IgNARs (Immunoglobulin New Antigen Receptors) are heavy chain-only Ig-like molecules that have been identified in all species of sharks studied so far. They are disulphide-linked homodimeric molecules composed of two polypeptide chains containing five constant domains and one variable region (VNAR) which binds antigens (Greenberg 1995).

VNARs are small (12 kDa), stable, soluble, monomeric antigen-binding domains that can be configured into many different therapeutic modalities. Owing to their elongated CDR3 structures that potentially extend into antigen clefts and cavities, VNARs are well suited to recognize epitopes on the external, apical domain of receptors on BBB endothelium that are conserved across species and do not interfere with endogenous ligand-receptor interactions.

Using a variety of in vitro and in vivo selection approaches, VNARs to TfR-1 have been identified that can shuttle therapeutic molecules across the brain capillary endothelium, which forms an impermeable blood-brain barrier (WO2016/077840; WO2018/031424; WO2019/089395; WO2020/056327). For example, VNARs to TfR-1 that function in vivo as effective BBB shuttles have remarkably different pharmacokinetic, potency and side-effect profiles than found with monoclonal antibodies to the same receptor.

Hence, there is a need for providing BBB shuttles that can deliver neuroprotective therapeutics, such as Trk agonist antibodies, into the brain at therapeutically efficacious doses. It would be desirable to combine a potent and selective BBB-shuttling VNAR domain, such as a human TfR-1 BBB shuttle, with one or more neurotrophic agonist antibodies (NAAb), such as Trk-selective antibodies, to produce a conjugate capable of uptake across a mammalian blood brain barrier (BBB) in a neuroprotective dose. This would be especially beneficial for a NAAb having one or more advantageous biological properties with therapeutic and/or diagnostic benefit in the BBB but which cannot to date be delivered across the BBB.

VNAR domains against BBB endothelial cell transporters, permits construction of potent and selective conjugates with TrkB agonist antibodies (TrkB-AAb).

### SUMMARY OF THE INVENTION

The present invention solves the problems described above by providing at least one BBB-shuttling VNAR domain operably linked to a neurotrophic agonist antibody (NAAb), said conjugate being capable of uptake across a mammalian blood brain barrier (BBB) in a neuroprotective effective amount. As exemplified herein, using VNAR domains against BBB endothelial cell transporters permits construction of potent and selective conjugates with NAAbs, including but not limited to, TrkA, TrkB and TrkC agonist antibodies (which are also referred to herein as TrkA-AAb, TrkB-AAb, and TrkC-AAb, respectively, and collectively referred to as “Trk agonist antibodies”).

Accordingly, one aspect of the invention provides a conjugate comprising at least one BBB-shuttling VNAR domain operably linked to a neurotrophic agonist antibody (NAAb), wherein the conjugate is capable of uptake across a mammalian blood brain barrier (BBB) in a therapeutically-effective amount. In some embodiments, the conjugate comprises a BBB-shuttling VNAR domain that is (a) a TfR-binding VNAR domain capable of specifically binding to a human TfR-1 without substantially interfering with transferrin binding to and/or transport by said human TfR-1, (b) a TfR-binding VNAR domain capable of specifically binding to a human TfR-1 without substantially interfering with transferrin binding to and/or transport by said human TfR-1 and capable of cross reacting with one or more of mouse, rat or non-human primate TfR-1, or (c) a TfR-binding VNAR domain capable of binding human TfR-1 with an EC50 ranging from about 1 nM to about 800 nM. In other embodiments, the BBB-shuttling VNAR domain binds to a membrane receptor capable of transcytosis across the BBB such as CD98hc.

In certain embodiments, the conjugate comprises a BBB-shuttling VNAR domain that is (a) the TfR-binding VNAR domain designated as Clone C or one of its variants, (b) the TfR-binding VNAR domain designated as Clone H or one of its variants, (c) the TfR-binding VNAR domain designated as Clone 8 or one of its variants, (d) TXB4, (e) a CD98-binding VNAR domain, (f) or any other TfR-binding VNAR domain disclosed in any of WO2016/077840; WO2018/031424; WO2019/089395; WO2019/246,288; WO2020/056327 or U.S. Ser. No. 63/112,314, filed Nov. 11, 2020. The foregoing and exemplary VNAR domains are more fully referenced in Table 1. Each such domain can be used independently, alone or in combination, in monovalent, bivalent or multivalent formats with the NAAb moiety of the conjugates.

In certain embodiments, the conjugate comprises a TrkA, TrkB or TrkC agonist antibody or an antigen binding fragment thereof. In some embodiments the conjugate comprises a TrkB agonist antibody or an antigen binding fragment thereof. In certain embodiments, the conjugate comprises the TrkB agonist antibody 29D7 or a chimeric, humanized or veneered version of monoclonal antibody 29D7 or an antigen-binding fragment of any of the foregoing.

In accordance with the invention, operable linkage of the at least one BBB-shuttling VNAR domain to the NAAb comprises one or more GlyGlyGlyGlySer (G4S) (SEQ ID NO. 1) units of amino acids. Other operable linkages known to those of skill in the art can also be used. In some embodiments, the conjugate comprises two or more independently selected BBB-shuttling VNAR domains. In certain embodiments, the conjugate comprises TXB4 operably linked to a TrkB agonist antibody. In certain embodiments the conjugate comprises or consists essentially of TrkB(HC2N) or TrkB(HV2N).

In some embodiments, the conjugate of the invention further comprises a diagnostic agent or another therapeutic agent.

Some aspects of the invention provide pharmaceutical compositions comprising a conjugate of the invention.

The instant invention also provides methods of treating neurodegenerative diseases or conditions which respond to activation of a neurotrophin receptor by administering a therapeutically-effective amount of a pharmaceutical composition of the invention to a mammalian subject in need thereof for a time and in an amount effective to treat the disease or condition. In some embodiments, composition is administered intravenously, intramuscularly, subcutaneously, intraarterially, intracranially or intrathecally, preferably, intravenously. Such delivery methods cause or lead to delivery of the conjugate to the brain of said mammalian subject and thereby treat the disease or condition. In certain embodiment the neurodegenerative disease or condition is Parkinson's disease, an acute or chronic neurological injury or wound, amyotrophic lateral sclerosis (ALS), or Alzheimer's disease (AD).

In another aspect, the invention provides a method of stimulating neuronal survival, growth, repair or regeneration which comprises contacting a population of neurons with a conjugate or pharmaceutical composition of the invention.

Further aspects of the invention relate to kits comprising the conjugate of the invention.

Yet further aspects of the invention relate to nucleic acids encoding the conjugates of the invention as well as vectors and host cells containing those nucleic acids and vectors.

### BRIEF DESCRIPTION OF THE DRAWINGS

FIG. 1. Models of Conjugates and Controls. This drawing is a schematic representation of a TrkB-AAb, and two genetic fusions of that antibody with a TfR-binding VNAR domain, TrkB(HC2N) and TrkB(HV2N), and a control VNAR-Fc fusion; all constructed as described in Example 1.

FIG. 2. Plasma Distribution of Conjugates. The unconjugated TrkB-AAb (circles), TrkB(HC2N) (squares) and TrkB(HV2N) (triangles) were administered intravenously to mice at 25 nmol/kg as described in Example 2. Plasma samples were collected after 0.5, 1, 2, 4 and 18 hours and analysed by ELISA (N=3, ±SD).

FIG. 3. Brain Uptake of Conjugates. The unconjugated TrkB-AAb (circles), TrkB(HC2N) (squares) and TrkB(HV2N) (triangles) were administered intravenously to mice at 25 nmol/kg as described in Example 2. Brains were excised after 0.5, 1, 2, 4 and 18 hours and homogenised before being analysed by ELISA (N=3, ±SD).

FIG. 4. Conjugates Retain Cross Species Reactivity. These graphs depict the binding curves for the TrkB conjugates and controls to human TfR-1 (hTfR1), mouse TfR-1 (mTfR1), rat TfR-1 (rTfR1) and cynomolgus TfR-1 (cTfR1) and human serum albumin (HSA) for unconjugated TrkB-AAb (solid circles), TrkB(HC2N) (squares), TrkB(HV2N) (triangles) and VNAR-Fc (open circles). A kinetic ELISA method was used to evaluate binding to each receptor with Vmax measured at 370 nm.

FIG. 5. Conjugates Retain Agonist Activity. Unconjugated TrkB antibody (open circles), HV2N (triangles) and HC2N (open squares) were tested for agonist activity in the TrkB-NFAT-bla CHO-K1 reporter cell line with BDNF (inverted triangles) as the positive control. Data are presented as the percentage of maximum BDNF response the mean (N=3, ±SEM).

FIG. 6. Prevention of 6-OHDA Lesions in the Striatum. TrkB(HV2N), designated as TrkB₁-TXB4 in this and the following figures, prevents development of 6-OHDA lesions in the striatum. Data represented as mean % TH immunoreactivity of contralateral striatum along the rostro-caudal axis (±SEM) as described in Example 4. The statistical difference for individual data points based on a paired two tailed t-test of TH+ immunoreactivity on the ipsilateral striatum relative to contralateral striatum for each brain area for the PBS control (circles) and TrkB(HV2N) (open circles) groups is not shown but each value in the TrkB(HV2N) group was significant at p<0.05 with none of the values in the control group being significant. The statistical difference between the size of the lesion across the rostral-caudal axis between the control and TrkB(HV2N) groups is shown and was p=0.003. The data points are mean of 3-5 animals per group with 3-6 measurements per animal made per region. TH, tyrosine hydroxylase.

FIG. 7. Prevention of 6-OHDA-induced Neuronal Loss in the SNc. TrkB(HV2N), prevents neuronal loss associated with 6-OHDA treatment. Data represented as mean % TH+ cell loss of contralateral SNc along the rostro-caudal axis (±SEM) as described in Example 4. The statistical difference for individual data points is based on a paired two tailed t-test of TH+ of cell count in the ipsilateral SNc relative to contralateral SNc for each brain area for the PBS control (circles) and TrkB(HV2N) (open circles) groups. The statistical difference between these groups (p=0.0179) is also indicated. The data points are mean of 3-5 animals per group with 3-6 measurements per animal made per region ***p=<0.0001, **p=<0.01, *p<0.05.

FIG. 8. Unconjugated TrkB Antibody Does Not Reduce Lesioning in the 6-OHDA Model. Data presented as combined mean (caudal+medial+rostral) of % TH immunoreactivity of contralateral striatum (±SEM) as described in Example 4. Paired two tailed t-test between PBS and unconjugated TrkB antibody was p=0.774. n=3-5 animals per group, with 2-6 measurements in each region for each animal.

FIG. 9. Unconjugated TrkB Antibody Does Not Protect Neurons in the 6-OHDA Model. Data presented as combined mean (caudal+medial+rostral) of % TH+ cell loss of the contralateral SNc as described in Example 4. Paired two tailed t-test between PBS and unconjugated TrkB antibody was p=0.249. n=3-5 animals per group with 2-6 measurements in each region for each animal.

### DETAILED DESCRIPTION OF THE INVENTION

In order that the present invention may be more readily understood, certain terms are defined below. Additional definitions may be found within the detailed description of the invention.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., cows, pigs), companion animals (e.g., dogs, cats) and rodents (e.g., mice and rats).

The term “non-human mammal” means a mammal which is not a human and includes, but is not limited to, a mouse, rat, rabbit, pig, cow, sheep, goat, dog, primate, or other non-human mammals typically used in research. As used herein, “mammals” includes the foregoing non-human mammals and humans.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment, and is not necessarily meant to imply complete cessation of the relevant disease, disorder or condition.

As used herein, the terms “preventing” and grammatical variants thereof refer to an approach for preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

As used herein, an “effective amount,” “therapeutically-effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

A physiologically-acceptable solution for use in an amount and for a time sufficient to effectively reduce a circulating concentration of the plurality of polypeptides is also referred to herein as a perfusate. The amount of perfusate and time of perfusion depends on the non-human mammal and can be readily determined by those of skill in the art. For example, with a mouse, using a volume of perfusate approximately 10x the blood volume of the mouse is effective at reducing the circulating concentration of polypeptides. Likewise, any volume of perfusate that reduces the circulating concentration of the plurality of polypeptides by about 10%, 25%, 50% or more (relative to the theoretical concentration of the plurality of polypeptides) being delivered is considered effective at reducing the circulating concentration of that plurality.

As used herein, a “VNAR domain” has the general structure, from N to C terminus, given by the formula FW1-CDR1-FW2-HV2-FW2′-HV4-FW3-CDR3-FW4, wherein the FWs are framework regions, CDRs are complementarity determining regions and HVs are hypervariable regions that form the variable domain of a shark IgNAR (“VNAR”). The CDR3 region in naturally-occurring VNARs is of heterogeneous size, ranging from about 7 to about 32 amino acid residues in length. The VNAR domains of the invention can optionally have a His-Tag (or other convenient tag for purification purposes). In some cases, such tags are removable. Typical VNAR domains have amino acid residues (aa) 1-25 of the framework 1 (FW1) region; aa 26-32 of the complimentary determining region 1 (CDR1); aa 33-43 of FW2; aa 44-52 of the hypervariable 2 region (HV2); aa 53-85 of FW3; aa 61-65 of HV4; the CDR3 region (of variable length) and FW4 (11 residues starting at XGXG).

As used herein, binding to the target of interest is called specific binding, while binding to other sites is called nonspecific binding. As used herein, a binding moiety, specific binding moiety, antibody or VNAR domain that “specifically binds” to its target does so selectively or preferentially. Such moieties, antibodies and VNARS can exhibit specific binding to multiple targets such as occurs when one of these entities exhibits species cross reactivity.

As used herein, the term “TfR,” “TfR1” or “TfR-1” refers to a mammalian transferrin receptor-1 (in context as a protein or a nucleic acid), unless the context indicates that it refers specifically to human TfR-1 (see, e.g., UniProt P02786 TFR1_Human) or mouse TfR-1.

The term “CD98 heavy chain” or “CD98hc” as used herein, refers to any native CD98hc from any vertebrate source, including mammals such as primates (e.g., humans) and rodents (e.g., mice and rats), unless otherwise indicated. CD98hc is also known by the names, inter alia, SLC3A2, 4F2, 4F2hc, Mdu1, Ly1O, Mdv1, Frp1, Mgp2, Mgp2hc, NACAE, 4T2, 4T2hc, and TROP4. The amino acid sequence of human CD98hc is provided in GenBank® Accession Nos. NP_001012680.1 (isoform b), NP_002385.3 (isoform c), NP_001012682.1 (e), and NP_001013269.1 (isoform f), respectively, and for murine CD98hc (“mCD98hc”), in GenBank® Accession No.: NP_001154885.1 (isoform a) and NP_032603.3 (isoform b)

Abbreviations used herein for conventional antibodies include: VL, variable region, light chain; VH, variable region, heavy chain; CL, light chain of; HC, heavy chain.

### Conjugates of the Invention

The present invention provides conjugates comprising at least one BBB-shuttling VNAR domain operably linked to a neurotrophic agonist antibody (NAAb) or a neurotrophic antagonist antibody, with the conjugate being capable of uptake across the BBB in therapeutically-effective amounts. Such conjugates are useful for treating neurological diseases and conditions responsive to modulation of neurotrophins.

Accordingly, one aspect of the invention is directed to conjugates comprising at least one BBB-shuttling VNAR domain operably linked to a neurotrophic agonist antibody (NAAb), with the conjugate being capable of uptake across the BBB in therapeutically-effective amounts. As used herein, a “BBB-shuttling VNAR domain” is a VNAR domain with binding specificity for a membrane transporter found on the endothelial or other cells of the BBB and which are capable of being endocytosed or transcytosed across the BBB in a manner that does not generally interfere with or impair the transport of the ligand binding and/or ligand transport properties of the transporter. Additionally, these VNAR domains can act as shuttles to transport other molecules (sometimes referred to as “cargo”) across the BBB and into the brain. Many such VNAR domains have been isolated and characterized which are further capable of delivering therapeutic levels of such cargo into the brain, including VNAR domains with specificity for TfR-1 and CD98, and many of those are more fully described below.

Thus, in some embodiments, the domains of the invention are BBB-shuttling VNAR domains that are independently one or more of (a) TfR-binding VNAR domains capable of specifically binding to a human TfR-1 without substantially interfering with transferrin binding to and/or transport by said human TfR-1, (b) TfR-binding VNAR domains capable of specifically binding to a human TfR-1 without substantially interfering with transferrin binding to and/or transport by said human TfR-1 and capable of cross reacting with mouse TfR 1, or (c) TfR-binding VNAR domains capable of binding human TfR-1 with an EC50 ranging from about 1 nM to about 800 nM.

In some embodiments, the domains of the invention are BBB-shuttling VNAR domains that are independently one or more of (a) the TfR-binding VNAR domains designated as Clone C or one of its variants described in WO2018/031424 and WO2019/089395, respectively; (b) the TfR-binding VNAR domains designated as Clone H or one of its variants described in WO2018/031424 and WO2019/089395, respectively; (c) the TfR-binding VNAR domains designated as Clone 8 or one of its variants described in WO2020/056327; (d) TXB4 (also known as Clone 18 and described in WO2020/056327; (e) the CD98-binding VNAR domains described in WO2020/246288; (0 the TfR-binding VNAR domains described in WO2016/077840 as capable of BBB shuttling; (g) the TfR-binding VNAR domain variants of Clone C specifically designated as variants 7, 13, 14, 16, 18, 25, 30, 31, and 34 in WO2019/089395; and (h) the VNAR-txp1 domain described in U.S. provisional application U.S. Ser. No. 63/112,314, filed Nov. 11, 2020. The amino acid sequences of these VNAR domains other than that in (h), have been published in the foregoing applications and expressly incorporated herein by reference. These and other specific VNAR domains suitable, independently alone or in combination, for use in the present conjugates are also provided in Table 1.

The TfR-binding VNAR domain designated here as TXB4 is a single domain shark antibody that binds to TfR-1 on these cells and can carry therapeutic antibodies across BBB to the brain parenchyma. The VNAR domain amino acid sequence for Clone TXB4 is:

(SEQ ID NO. 2)
ARVDQTPQTITKETGESLTINCVLRDSNCALSSTYWYRKKSGSTNEENI
SKGGRYVETVNSGSKSFSLRINDLTVEDSGTYRCNVVQYPQYPNYFWCD
VYGDGTAVTVNA.

Clone C is a human and mouse TfR-binding VNAR obtained by in vivo selection of brain penetrating phages as described in Examples 1 and 2 of Intl. Appln. No. PCT/US2017/045592, filed Aug. 4, 2017 (now WO2018/031424). The VNAR domain amino acid sequence for Clone C is:

(SEQ ID NO. 3)
ARVDQTPQTITKETGESLTINCVLRDSNCALSSTYWYRKKSGSTNEENI
SKGGRYVETVNSGSKSFSLRINDLTVEDSGTYRCNVVQYPSYNNYFWCD
VYGDGTAVTVN.

Suitable variants of Clone C are described in WO2019/098395 and in Table 1.

Clone H is a human and mouse TfR-binding VNAR obtained by in vivo selection of brain penetrating phages as described in Examples 1 and 2 of Intl. Appln. No. PCT/US2017/045592, filed Aug. 4, 2017 (now WO2018/031424). The VNAR domain amino acid sequence for Clone H is:

(SEQ ID NO. 4)
ARVDQTPQTITKETGESLTINCVLRDSNCELSSTYWYRKKSGSTNEESI
SKGGRYVETVNSGSKSFSLRINDLVVEDSGTYRCNVQQFPSSSNGRYWC
DVYGGGTAVTVNA.

Suitable variants of Clone H are described in WO2019/098395 and in Table 1.

Clone 8 described in WO2020/056327 is a human TfR-1 binding VNAR. The VNAR domain amino acid sequence for Clone 8 is:

(SEQ ID NO. 5)
ARVDQTPQTITKETGESLTINCVLRDSNCALPSTYWYRKKSGSTNEESI
SKGGRYVETVNSGSKSFSLRINDLTVEDSGTYRCKVIAQLSSILRGCNY
RKHDVYGDGTAVTVNA.

Suitable variants of Clone 8 are described in WO2020/056327 and in Table 1.

The VNAR domain designated as VNAR-txp1 binds to human and macaque TfR-1 and is described in U.S. Ser. No. 63/112,314, filed Nov. 11, 2020. The amino acid sequence of VNAR-txp1 is

(SEQ ID NO. 6)
ARVDQTPQTITKETGESLTINCVLRDSNCALSSTYWYRKKSGSTNEENI
SKGGRYVETVNSGSKSFSLKINDLTVEDSGTYRCNVVGTWCMSWRDVYG
GGTAVTVNA.

Clone F12 is a Type IV VNAR domain capable of specifically binding to human and murine CD98hc as described in WO2019/246288. The VNAR domain amino acid sequence for Clone F12 is:

(SEQ ID NO. 7)
ARVDQTPQTITKEEGESLTINCVLRVHGRALASTSWYRKKSGSTREETI
SKGGRYVETVNSGSKSFSLRINDLTVEDSGTYRCNVYGLSFGDIEGVKK
IDVYGDGTAVTVNA.

Suitable variants of Clone F12 and other CD98hc-binding VNARs are described in WO2019/246288 and in Table 1.

TABLE 1
Exemplary BBB-shuttling VNARs

| | | | Identifying | |
|---|---|---|---|---|
| | | | SEQ ID | |
| | | | NO. in | Type of |
| Name | Target | Publication | Publication | Sequence |
| Clone C | TfR-1 | WO2018/031424 | 10, 164 | domain |
| Clone 1 | TfR-1 | WO2018/031424 | 1 | domain |
| Clone 2 | TfR-1 | WO2018/031424 | 2 | domain |
| Clone 7 | TfR-1 | WO2018/031424 | 7 | domain |
| Clone 11 | TfR-1 | WO2018/031424 | 11 | domain |
| Clone 12 | TfR-1 | WO2018/031424 | 12 | domain |
| Clone 39 | TfR-1 | WO2018/031424 | 39 | domain |
| Clone H | TfR-1 | WO2018/031424 | 169 | domain |
| Clone C var. 13 | TfR-1 | WO2019/089395 | 26 | CDR3 |
| Clone C var. 18 | TfR-1 | WO2019/089395 | 31 | CDR3 |
| Clone C var. 38 | TfR-1 | WO2019/089395 | 38 | CDR3 |
| Clone H var. 1 | TfR-1 | WO2019/089395 | 55 | CDR3 |
| Clone H var. 10 | TfR-1 | WO2019/089395 | 64 | CDR3 |
| F1 | CD98hc | WO2019/246288 | 7 | domain |
| E12 | CD98hc | WO2019/246288 | 13 | domain |
| F12 | CD98hc | WO2019/246288 | 20 | domain |
| F12-A01 | CD98hc | WO2019/24628 8 | 98 | CDR3 |
| F12-B12 | CD98hc | WO2019/24628 8 | 99 | CDR3 |
| F12-C01 | CD98hc | WO2019/24628 8 | 100 | CDR3 |
| F12-E03 | CD98hc | WO2019/24628 8 | 101 | CDR3 |
| F12-G02 | CD98hc | WO2019/24628 8 | 102 | CDR3 |
| F12-G04 | CD98hc | WO2019/24628 8 | 103 | CDR3 |
| F12-H04 | CD98hc | WO2019/24628 8 | 104 | CDR3 |
| Clone 5 | TfR-1 | WO2020/056327 | 18 | domain |
| Clone 8 | TfR-1 | WO2020/056327 | 7 | domain |
| Clone 10 | TfR-1 | WO2020/056327 | 22 | domain |
| Clone 22 | TfR-1 | WO2020/056327 | 34 | domain |
| Clone 8 | TfR-1 | WO2020/056327 | 10 | CDR3 |
| Clone 8.5 | TfR-1 | WO2020/056327 | 11 | CDR3 |
| Clone 8.8 | TfR-1 | WO2020/056327 | 12 | CDR3 |
| Clone 8.12 | TfR-1 | WO2020/056327 | 13 | CDR3 |

In accordance with the invention, the conjugates of the invention comprise a neurotrophic agonist antibody. As used herein “neurotrophic agonist antibody” or “NAAb ” means an antibody (or antigen binding fragment thereof) which upon binding of a neurotrophin receptor activates the biological activity of that receptor in a manner similar to the activation of that receptor by its cognate neurotrophin but not necessarily to the same degree or level of activation. In some embodiments, the NAAb is a TrkA, TrkB or TrkC agonist antibody (i.e., a TrkA-AAb, TrkB-AAb, and TrkC-AAb, respectively) or an antigen binding fragment thereof. In some embodiments the NAAb is a TrkB agonist antibody or an antigen binding fragment thereof. In some embodiments the TrkB-AAb is murine monoclonal antibody 29D7 or any chimeric, humanized or veneered version thereof. The conjugate of claim 8, wherein said conjugate is TXB4 operably linked to a TrkB agonist antibody.

The NAAbs of the invention include, but are not limited to, those described in U.S. Pat. No. 7,750,122; Szobota 2019; Han 2019; Merkouris 2018; Kim 2014; Rosenthal 2014; Todd 2014; Perreault 2013; Vugmeyster 2013; Cazorla 2011; Fouad 2010; Sahenk 2010; Bai 2010; Xu 2010; Hu 2010; and Qian 2006; and/or any chimeric, humanized or veneered version thereof or antigen binding fragment thereof.

In accordance with the invention, the VNAR domains and NAAbs of the invention are operably linked by chemical or biological linkages known in the art. In one embodiment, the VNAR domain and NAAb are operably linked by one or more GlyGlyGlyGlySer (G4S) units of amino acids (SEQ ID NO. 1). The number of such units can range from 1 to 10 and can be determined by those of skill in the art.

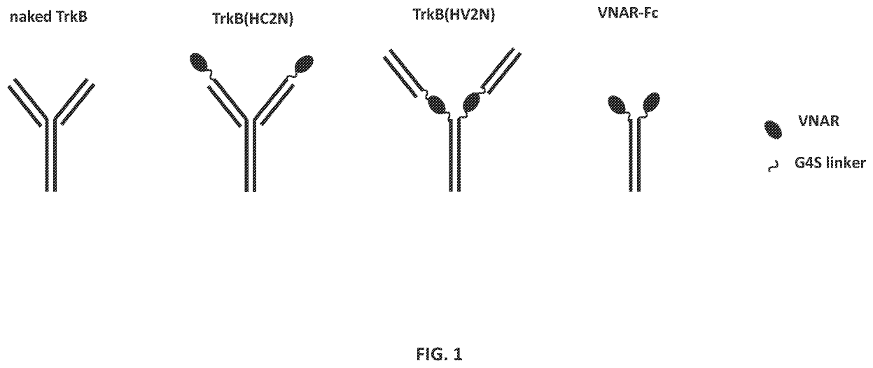

Embodiments of the conjugates of the invention comprise at least one, and preferably two or more independently selected BBB-shuttling VNAR domains. The location of these domains can be on the N or C terminus of the NAAb (independently located on light or heavy chains) or at an internal location in the NAAb, provided such linkage does not disrupt the agonist activity of the NAAb or the binding activity of the VNAR domain. Examples of arrangements of VNARs and NAAbs are shown in FIG. 1.

In one preferred embodiment, the conjugate of the invention comprises TrkB(HC2N) or TrkB(HV2N) which are more fully described in Example 1. In one preferred embodiment of the invention, the conjugate of the invention is TrkB(HC2N) or TrkB(HV2N)

Associated additional diagnostic or therapeutic (“heterologous”) molecules which may be used in conjunction with any one of the above embodiments may comprise, e.g., one or more biologically active molecules and/or imaging agents. Exemplary polypeptides which may be therapeutically beneficial when administered in combination with a neurotrophic agonist antibody (e.g., a Trk-selective antibody) of the invention include but are not limited to: a brain derived neurotrophic factor (BDNF), a bone morphogenic protein (e.g., BMP-1 through BMP-7, BMP8a, BMP8b, BMP10 and BMP15), a ciliary neurotrophic factor (CNF), an epidermal growth factor (EGF), erythropoietin, a fibroblast growth factor (FGF), a glial derived neurotrophic factor (GDNF), a heptocyte growth factor, an interleukin (e.g., IL-1, IL-4, IL-6, IL-10, IL-12, IL-13, IL-15, IL-17), a nerve growth factor (NGF), a neurotrophin (e.g., NT-3 and NT-4/5), a neurturin, a neuregulin, a platelet derived growth factor (PDGF), a transforming growth factor (e.g., TGF-alpha and TGF-beta), apolipoprotein E (ApoE), a vasoactive intestinal peptide, artemin, persephin, netrin, neurotensin, GM-GSF, cardiotrophin-1, stem cell factor, midkine, pleiotrophin, a saposin, a semaporin, leukemia inhibitory factor, and the like.

Other exemplary biologically active molecules which may be transported include, e.g., toxins for targeted cell death (useful e.g., in certain hyperproliferative diseases or disorders such as cancers or aberrant proliferative conditions). Other exemplary biologically active molecules which may be transported in association with a conjugate of the invention include, e.g., polypeptides, such as an antibody or antibody fragment; a therapeutic peptide such as a hormone, cytokine, growth factor, enzyme, antigen or antigenic peptide, transcription factor, or any functional domain thereof. Other exemplary biologically active molecules which may be transported include, e.g., nucleic acid molecules, such as an oligonucleotide (e.g., single, double or more stranded RNA and/or DNA molecules, and analogs and derivatives thereof); small regulatory RNA such as shRNA, miRNA, siRNA and the like; and a plasmid or fragment thereof.

Exemplary embodiments of an imaging agent as an associated heterologous molecule include agents that comprise at least one of a metal such as a paramagnetic metal, a radionuclide such as a radioisotope, a fluorochrome or fluorophor, an energy emitting particle, a detectable dye, and an enzyme substrate.

Numerous other examples of biologically active molecules may be used in association with the conjugates of the invention, appropriate selection of which will be apparent to the skilled artisan depending on the condition, disease or disorder to be treated.

An imaging agent, as used herein, may be any chemical substance which may be used to provide a signal or contrast in imaging. A signal enhancing domain may be an organic molecule, metal ion, salt or chelate, a particle (e.g., iron particle), or a labeled peptide, protein, glycoprotein, polymer or liposome. For example, an imaging agent may include one or more of a radionuclide, a paramagnetic metal, a fluorochrome, a dye, and an enzyme substrate.

For x-ray imaging, the imaging agent may comprise iodinated organic molecules or chelates of heavy metal ions of atomic numbers 57 to 83. In certain embodiments, the imaging agent is I¹²⁵ labeled IgG (see, e.g., M. Sovak, ed., “Radiocontrast Agents,” Springer-Verlag, pp. 23-125 (1984).

For ultrasound imaging, an imaging agent may comprise gas-filled bubbles or particles or metal chelates where the metal ions have atomic numbers 21-29, 42, 44 or 57-83. See e.g., Tyler et al., Ultrasonic Imaging, 3, pp. 323-29 (1981) and D. P. Swanson, “Enhancement Agents for Ultrasound: Fundamentals,” Pharmaceuticals in Medical Imaging, pp. 682-87. (1990) for other suitable compounds.

For nuclear radiopharmaceutical imaging or radiotherapy, an imaging agent may comprise a radioactive molecule. In certain embodiments, chelates of Tc, Re, Co, Cu, Au, Ag, Pb, Bi, In and Ga may be used. In certain embodiments, chelates of Tc-99m may be used. See e.g., Rayudu GVS, Radiotracers for Medical Applications, I, pp. 201 and D. P. Swanson et al., ed., Pharmaceuticals in Medical Imaging, pp. 279-644 (1990) for other suitable compounds.

For ultraviolet/visible/infrared light imaging, an imaging agent may comprise any organic or inorganic dye or any metal chelate.

For MRI, an imaging agent may comprise a metal-ligand complex of a paramagnetic form of a metal ion with atomic numbers 21-29, 42, 44, or 57-83. In certain embodiments, the paramagnetic metal is selected from: Cr(III), Cu(II), Dy(III), Er(III) and Eu(III), Fe(III), Gd(III), Ho(III), Mn(II and III), Tb(III). A variety of chelating ligands useful as MRI agents are well known in the art.

In sum, the invention provides BBB-shuttling VNAR domains operably linked to a NAAb. In certain embodiments, the invention provides a TfR-specific conjugate comprising a TfR-specific VNAR domain of the invention operably linked to a Trk-selective antibody, including but not limited to TrkA, TrkB and/or TrkC antibodies that are agonistic (i.e., which stimulate Trk signaling) or antagonistic (i.e., which reduce Trk signaling).

Nucleic Acid Sequences that Encode Conjugates of the Invention

In one aspect, the invention provides an isolated nucleic acid which encodes a conjugate of the invention, or a fragment or derivative thereof. The invention also provides an isolated nucleic acid molecule comprising a sequence that hybridizes under stringent conditions to a nucleic acid sequence which encodes a conjugate of the invention, or a fragment or derivative thereof, or the antisense or complement of any such sequence.

In another aspect, the invention provides an isolated nucleic acid molecule encoding a fusion protein comprising at least two segments, wherein one of the segments comprises a conjugate of the invention. In certain embodiments, a second segment comprises a heterologous signal polypeptide, a heterologous binding moiety, an immunoglobulin fragment such as a Fc domain, or a detectable marker.

One aspect of the invention provides isolated nucleic acid molecules that encode the conjugates of the invention. Also included are nucleic acid fragments sufficient for use as hybridization probes to identify conjugates of the invention and fragments for use as polymerase chain reaction (PCR) primers for the amplification or mutation of conjugate-encoding nucleic acid molecules.

As used herein, the term “nucleic acid molecule” is intended to include DNA molecules, RNA molecules (e.g., mRNA, shRNA, siRNA, microRNA), analogs of the DNA or RNA generated using nucleotide analogs, and derivatives, fragments and homologs thereof. The nucleic acid molecules of the invention may be single-, double-, or triple-stranded. A nucleic acid molecule of the present invention may be isolated using sequence information provided herein and well known molecular biological techniques (e.g., as described in Sambrook et al., Eds., MOLECULAR CLONING: A LABORATORY MANUAL 2ND ED., Cold Spring Harbor Laboratory Press, Cold Spring Harbor, N.Y., 1989; and Ausubel, et al., Eds., CURRENT PROTOCOLS IN MOLECULAR BIOLOGY, John Wiley & Sons, New York, N.Y., 1993).

A nucleic acid molecule of the invention may be amplified using any form of nucleic acid template and appropriate oligonucleotide primers according to standard PCR amplification techniques. Amplified nucleic acid may be cloned into an appropriate vector and characterized, e.g., by restriction analysis or DNA sequencing. Furthermore, oligonucleotides corresponding to nucleotide sequences that encode a TfR selective binding moiety or compound of the invention may be prepared by standard synthetic techniques, e.g., using an automated DNA synthesizer.

The term “oligonucleotide” as used herein refers to a series of covalently linked nucleotide (or nucleoside residues, including ribonucleoside or deoxyribonucleoside residues) wherein the oligonucleotide has a sufficient number of nucleotide bases to be used in a PCR reaction. Oligonucleotides comprise portions of a nucleic acid sequence having at least about 10 nucleotides and as many as 50 nucleotides, preferably about 15 nucleotides to 30 nucleotides. Oligonucleotides may be chemically synthesized and may be used as probes. A short oligonucleotide sequence may be used to amplify, confirm, or reveal the presence of an identical, similar or complementary DNA or RNA in a particular cell or tissue.

Derivatives or analogs of the nucleic acid molecules (or proteins) of the invention include, inter alia, nucleic acid (or polypeptide) molecules having regions that are substantially homologous to the nucleic acid molecules or proteins of the invention, e.g., by at least about 45%, 50%, 70%, 80%, 95%, 98%, or even 99% identity (with a preferred identity of 80-99%) over a nucleic acid or amino acid sequence of the same size or when compared to an aligned sequence in which the alignment is done by a computer homology program known in the art. A percent identity for any candidate nucleic acid or polypeptide relative to a reference nucleic acid or polypeptide may be determined by aligning a reference sequence to one or more test sequences using, for example, the computer program ClustalW (version 1.83, default parameters), which enable nucleic acid or polypeptide sequence alignments across their entire lengths (global alignment) or across a specified length. The number of identical matches in such a ClustalW alignment is divided by the length of the reference sequence and multiplied by 100.

Also included are nucleic acid molecules capable of hybridizing to the complement of a sequence encoding the conjugates of the invention under stringent or moderately stringent conditions. See e.g. Ausubel, et al., CURRENT PROTOCOLS IN MOLECULAR BIOLOGY, John Wiley & Sons, New York, N.Y., 1993, and below. An exemplary program is the GAP program (Wisconsin Sequence Analysis Package, Version 8 for UNIX, Genetics Computer Group, University Research Park, Madison, Wis.) using the default settings, which uses the algorithm of Smith and Waterman (1981) Adv. Appl. Math. 2:482489). Derivatives and analogs may be full length or other than full length, if the derivative or analog contains a modified nucleic acid or amino acid, as described below.

Stringent conditions are known to those skilled in the art and may be found in CURRENT PROTOCOLS IN MOLECULAR BIOLOGY, John Wiley & Sons, N.Y. (1989), 6.3.1-6.3.6. In certain embodiments, stringent conditions typically permit sequences at least about 65%, 70%, 75%, 85%, 90%, 95%, 98%, or 99% homologous to each other to remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. The term “stringent hybridization conditions” as used herein refers to conditions under which a nucleic acid probe, primer or oligonucleotide will hybridize to its target sequence, but only negligibly or not at all to other nucleic acid sequences. Stringent conditions are sequence- and length-dependent, and depend on % (percent)-identity (or %-mismatch) over a certain length of nucleotide residues. Longer sequences hybridize specifically at higher temperatures than shorter sequences. Generally, stringent conditions are selected to be about 5° C. lower than the thermal melting point (Tm) for the specific sequence at a defined ionic strength and pH. Stringent conditions may also be achieved with the addition of destabilizing agents, such as formamide.

### Methods of Producing Conjugates of the Invention

The conjugates of the invention may be manufactured by standard synthetic methods, by use of recombinant expression systems, or by any other suitable method. Thus, the conjugates may be synthesized in a number of ways, including, e.g., methods comprising: (1) synthesizing the VNAR domain using standard solid-phase or liquid-phase methodology, either stepwise or by fragment assembly, and isolating and purifying the domain and covalently or non-covalently linking it to already purified NAAb; (2) expressing a nucleic acid construct that encodes VNAR or conjugate component in a host cell and recovering the expression product from the host cell or host cell culture; or (3) cell-free in vitro expression of a nucleic acid construct encoding a conjugate of the invention, and recovering the expression product; or by any combination of the methods of (1), (2) or (3) to obtain fragments of the peptide component, subsequently joining (e.g., ligating) the fragments to obtain the conjugate, and recovering the conjugate.

Accordingly, the present invention also provides methods for producing a conjugate of the invention according to above recited methods; a nucleic acid molecule encoding part or all of a conjugate of the invention, a vector comprising at least one nucleic acid of the invention, expression vectors comprising at least one nucleic acid of the invention capable of producing a conjugate of the invention when introduced into a host cell, and a host cell comprising a nucleic acid molecule, vector or expression vector of the invention.

Conjugates of the invention may be prepared using recombinant techniques well known in the art. In general, methods for producing polypeptides by culturing host cells transformed or transfected with a vector comprising the encoding nucleic acid and recovering the polypeptide from cell culture are described in, e.g., Sambrook et al., Molecular Cloning: A Laboratory Manual (New York: Cold Spring Harbor Laboratory Press, 1989); Dieffenbach et al., PCR Primer: A Laboratory Manual (Cold Spring Harbor Laboratory Press, 1995).

A nucleic acid encoding a desired polypeptide may be inserted into a replication vector for further cloning (amplification) of the DNA or for expression of the nucleic acid into RNA and protein. A multitude of cloning and expression vectors are publicly available.

Expression vectors capable of directing transient or stable expression of genes to which they are operably linked are well known in the art. The vector components generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

Any suitable host cell may be used to produce conjugates of the invention. Host cells may be cells stably or transiently transfected, transformed, transduced or infected with one or more expression vectors which drive expression of a polypeptide of the invention. Suitable host cells for cloning or expressing nucleic acids of the invention include prokaryote, yeast, or higher eukaryote cells. Eukaryotic microbes such as filamentous fungi yeast, Arabidopsis, and other plant and animal eukaryotic host cells that may be grown in liquid culture are suitable cloning or expression hosts for vectors. Suitable host cells for the expression of glycosylated polypeptides may also be derived from multicellular organisms.

Creation and isolation of host cell lines producing a conjugate of the invention can be accomplished using standard techniques known in the art. Mammalian cells are preferred host cells for expression of peptides. Particularly useful mammalian cells include, inter alia, HEK 293, NSO, DG-44, and CHO cells, but any other suitable host cell may be used according to the invention. Preferably, the conjugates are secreted into the medium in which the host cells are cultured, from which the conjugates may be recovered or purified.

When a polypeptide is produced in a recombinant cell other than one of human origin, it is typically free of polypeptides of human origin. In certain embodiments, it is advantageous to separate a polypeptide away from other recombinant cell components such as host cell polypeptides to obtain preparations that are of high purity or substantially homogeneous. As a first step, culture medium or cell lysates may be centrifuged to remove particulate cell debris and suitable protein purification procedures may be performed. Such procedures include, inter alia, fractionation (e.g., size separation by gel filtration or charge separation by ion-exchange column); ethanol precipitation; Protein A Sepharose columns to remove contaminants such as IgG; hydrophobic interaction chromatography; reverse phase HPLC; chromatography on silica or on cation-exchange resins such as DEAE and the like; chromatofocusing; electrophoretic separations; ammonium sulfate precipitation; gel filtration using, for example, Sephadex beads such as G-75. Any number of biochemical purification techniques may be used to increase the purity of a conjugate of the invention.

### Methods of Treatment Using Conjugates of the Invention

The present invention provides conjugates for use, alone or in combination with one or more additional therapeutic agents, in a pharmaceutical composition, for treatment or prophylaxis of a neurodegenerative disease or a condition which responds to activation of a neurotrophin receptor. The method comprises administering a therapeutically-effective amount of a conjugate or pharmaceutical composition comprising a conjugate of the invention to a mammalian subject in need thereof for a time and in an amount effective to treat the disease or condition.

In some embodiments, the conjugates of the invention are administered intravenously, intramuscularly, subcutaneously, intraarterially, intracranially or intrathecally. In an embodiment, the conjugate is preferably administered intravenously. The routes of administration can be determined by those of skill in the art and lead to accumulation of the conjugate in the brain of the mammalian subject to thereby treat the disease or condition.

The methods of the invention can be used to treat neurodegenerative diseases or conditions that include but are not limited to, Parkinson's disease, an acute or chronic neurological injury or wound, amyotrophic lateral sclerosis (ALS), or Alzheimer's disease (AD).

In some embodiments, the conjugate comprises two BBB-shuttling VNAR domains that bind human TfR-1 operably linked to a TrkB agonist antibody or an antigen binding fragment thereof. In some embodiments of the method, that conjugate is TXB4 operably linked to a TrkB agonist antibody, and in other embodiments, that conjugate comprises or is TrkB(HC2N) or TrkB(HV2N.

In accordance with the foregoing, the conjugates of the invention can be used the preparation of a medicament to treat a neurodegenerative disease or condition in a mammalian subject in need thereof.

In another aspect, the instant invention is drawn to a method of stimulating neuronal survival, growth, repair or regeneration which comprises contacting a population of neurons with a conjugate of the invention.

In yet other aspects, the conjugates of the invention are effective in treating a brain or CNS disease, condition, injury or disorder, such as, for example, neurodegenerative diseases, neuronal injury, stroke, genetic disorders, psychiatric disorders, developmental disorders, inflammation, infection or damage, and brain cancers, spinal cord injury (SCI) and traumatic brain injury (TBI). In certain embodiments, a brain disorder is selected from epilepsy, meningitis, encephalitis including HIV Encephalitis, progressive multifocal leukoencephalopathy, neuromyelitis optica, multiple sclerosis, late-stage neurological trypanosomiasis, amyotrophic lateral sclerosis (ALS), progressive bulbar palsy (PBP), primary lateral sclerosis (PLS), progressive muscular atrophy (PMA), Alzheimer's disease, Parkinson's disease, Huntington's disease, De Vivo disease, and any type of tumor, cancer or hyperproliferative disease in the brain or CNS.

### Pharmaceutical Compositions, Administration and Dosing

The present invention further provides pharmaceutical compositions comprising a conjugate of the invention, or a pharmaceutically acceptable salt or solvate thereof, according to the invention, together with a pharmaceutically acceptable carrier, excipient or vehicle. Certain embodiments of the pharmaceutical compositions of the invention are described in further detail below.

Conjugates of the present invention, or salts thereof, may be formulated as pharmaceutical compositions prepared for storage or administration, which typically comprise a therapeutically effective amount of a compound of the invention, or a salt thereof, in a pharmaceutically acceptable carrier.

The therapeutically effective amount of a conjugate of the present invention will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. pH buffering agents may be phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. The term further encompasses any agents listed in the US Pharmacopeia for use in animals, including humans.

The term “pharmaceutically-acceptable salt” refers to the salt of the compounds. As used herein a pharmaceutically-acceptable salt retains qualitatively a desired biological activity of the parent compound without imparting any undesired effects relative to the compound. Salts include pharmaceutically acceptable salts such as acid addition salts and basic salts. Acid addition salts include salts derived from nontoxic inorganic acids, such as hydrochloric, nitric, phosphorous, phosphoric, sulfuric, hydrobromic, hydroiodic and the like, or from nontoxic organic acids such as aliphatic mono- and di-carboxylic acids, phenyl-substituted alkanoic acids, hydroxy alkanoic acids, aromatic acids, aliphatic and aromatic sulfonic acids and the like. Examples of basic salts include salts where the cation is selected from alkali metals, such as sodium and potassium, alkaline earth metals such as calcium and magnesium, and ammonium ions ⁺N(R³)₃(R⁴), where R³ and R⁴ independently designate optionally substituted C₁₋₆-alkyl, optionally substituted C₂₋₆-alkenyl, optionally substituted aryl, or optionally substituted heteroaryl, and more specifically, the organic amines, such as N, N′-dibenzylethylenediamine, N-methylglucamine, chloroprocaine, choline, diethanolamine, ethylenediamine, procaine and the like. Other examples of pharmaceutically acceptable salts are described in “Remington's Pharmaceutical Sciences”, 17th edition. Ed. Alfonso R. Gennaro (Ed.), Mark Publishing Company, Easton, Pa., U.S.A., 1985 and more recent editions, and in the Encyclopaedia of Pharmaceutical Technology.

“Treatment” is an approach for obtaining beneficial or desired clinical results. For the purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures in certain embodiments. Those in need of treatment include those already with the disorder as well as those in which the disorder is to be prevented. By treatment is meant inhibiting or reducing an increase in pathology or symptoms when compared to the absence of treatment, and is not necessarily meant to imply complete cessation of the relevant condition.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration.

Pharmaceutically acceptable carriers or diluents include those used in formulations suitable for oral, rectal, nasal or parenteral (including subcutaneous, intramuscular, intravenous, intradermal, and transdermal) administration. The formulations may conveniently be presented in unit dosage form and may be prepared by any of the methods well known in the art of pharmacy. Subcutaneous or transdermal modes of administration may be particularly suitable for the compounds described herein.

An acceptable route of administration may refer to any administration pathway known in the art, including but not limited to aerosol, enteral, nasal, ophthalmic, oral, parenteral, rectal, vaginal, or transdermal (e.g., topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

In another aspect, the present invention provides a composition, e.g., a pharmaceutical composition, comprising one or a combination of different conjugates of the invention and at least one pharmaceutically acceptable carrier.

Pharmaceutical compositions of the invention may be administered alone or in combination with one or more other therapeutic or diagnostic agents. A combination therapy may include a conjugate of the present invention combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutics which may complement or otherwise be beneficial in a therapeutic or prophylactic treatment regimen.

As used herein, “pharmaceutically acceptable carrier” includes any and all physiologically acceptable, i.e., compatible, solvents, dispersion media, coatings, antimicrobial agents, isotonic and absorption delaying agents, and the like. In certain embodiments, the carrier is suitable for intravenous, intramuscular, subcutaneous, parenteral, spinal or epidermal administration (e.g., by injection or infusion). Depending on selected route of administration, the conjugate of the invention may be coated in a material or materials intended to protect the compound from the action of acids and other natural inactivating conditions to which the active conjugate may encounter when administered to a subject by a particular route of administration.

A pharmaceutical composition of the invention also optionally includes a pharmaceutically acceptable antioxidant. Exemplary pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants.

Composition comprising conjugates of the invention may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

Exemplary pharmaceutically acceptable carriers include sterile aqueous solutions or dispersions and sterile powders for the extemporaneous preparation of sterile injectable solutions or dispersion. Such media and reagents for pharmaceutically active substances are known in the art. The pharmaceutical compositions of the invention may include any conventional media or agent unless any is incompatible with the active conjugate of the invention. Supplementary active compounds may further be incorporated into the compositions.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g., glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In certain embodiments, isotonic agents, e.g., sugars, polyalcohols such as mannitol, sorbitol, or sodium chloride may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a conjugate of the invention in the required amount in an appropriate solvent with one or a combination of ingredients described above, as required, followed by sterilization microfiltration. Dispersions may be prepared by incorporating the active compound into a sterile vehicle that contains dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of a conjugate of the invention is administered by, e.g., intravenous, cutaneous or subcutaneous injection, the binding agent will be in the form of a pyrogen-free, parenterally acceptable aqueous solution. Methods for preparing parenterally acceptable protein solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or other vehicle as known in the art. A pharmaceutical composition of the present invention may also contain stabilizers, preservatives, buffers, antioxidants, or other additives well known to those of skill in the art.

The amount of active ingredient which can be combined with a carrier material to produce a single dosage form will vary depending on a variety of factors, including the subject being treated, and the particular mode of administration. In general, it will be an amount of the composition that produces an appropriate therapeutic effect under the particular circumstances. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens may be adjusted to provide the optimum desired response (e.g., a therapeutic response). For example, a single bolus may be administered, several divided doses may be administered over time, or the dose may be proportionally reduced or increased as indicated by the particular circumstances of the therapeutic situation, on a case by case basis. It is especially advantageous to formulate parenteral compositions in dosage unit forms for ease of administration and uniformity of dosage when administered to the subject or patient. As used herein, a dosage unit form refers to physically discrete units suitable as unitary dosages for the subjects to be treated; each unit containing a predetermined quantity of active compound calculated to produce a desired therapeutic effect in association with the required pharmaceutical carrier. The specification for the dosage unit forms of the invention depend on the specific characteristics of the active compound and the particular therapeutic effect(s) to be achieved, taking into consideration and the treatment and sensitivity of any individual patient.

For administration of a conjugate of the invention, the dosage range will generally be from about 0.0001 to 100 mg/kg, and more usually 0.01 to 5 mg/kg, of the host body weight. Exemplary dosages may be 0.25 mg/kg body weight, 1 mg/kg body weight, 3 mg/kg body weight, 5 mg/kg body weight or 10 mg/kg body weight or within the range of 1-10 mg/kg. An exemplary treatment regime is a once or twice daily administration, or a once or twice weekly administration, once every two weeks, once every three weeks, once every four weeks, once a month, once every two or three months or once every three to 6 months. Dosages may be selected and readjusted by the skilled health care professional as required to maximize therapeutic benefit for a particular subject, e.g., patient. Conjugates will typically be administered on multiple occasions. Intervals between single dosages can be, for example, 2-5 days, weekly, monthly, every two or three months, every six months, or yearly. Intervals between administrations can also be irregular, based on monitoring blood levels of conjugates of the invention in a subject or patient. In some methods, dosage is adjusted to achieve a plasma concentration known to give suitable brain concentrations of the conjugate. Such a plasma level may range from about 1-1000 μg/ml and in some methods about 25-300 μg/ml. Dosage regimens for a conjugates of the invention include intravenous administration of 1 mg/kg body weight or 3 mg/kg body weight with the compound administered every two to four weeks for six dosages, then every three months at 3 mg/kg body weight or 1 mg/kg body weight.

In certain embodiments, two or more conjugates of the invention with different binding properties may be administered simultaneously or sequentially, in which case the dosage of each administered compound may be adjusted to fall within the ranges described herein.

In certain embodiments, a conjugate of the invention may be administered as a sustained release formulation, in which case less frequent administration is required. Dosage and frequency vary depending on the half-life of the conjugate in the subject or patient. The dosage and frequency of administration may vary depending on whether the treatment is therapeutic or prophylactic (e.g., preventative), and may be adjusted during the course of treatment. In certain prophylactic applications, a relatively low dosage is administered at relatively infrequent intervals over a relatively long period of time. Some subjects may continue to receive treatment over their lifetime. In certain therapeutic applications, a relatively high dosage at relatively short intervals is sometimes required until progression of the disease is reduced or until the patient shows partial or complete amelioration of symptoms of disease. Thereafter, the patient may be switched to a suitable prophylactic dosing regimen.

Actual dosage levels of the conjugate alone or in combination with one or more other active ingredients in the pharmaceutical compositions of the present invention may be varied so as to obtain an amount of the active ingredient which is effective to achieve the desired therapeutic response for a particular patient, composition, and mode of administration, without causing deleterious side effects to the subject or patient. A selected dosage level will depend upon a variety of factors, such as pharmacokinetic factors, including the activity of the particular conjugate or composition employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject or patient being treated, and similar factors well known in the medical arts.

Administration of a “therapeutically effective dosage” of a conjugate of the invention may result in a decrease in severity of disease symptoms, an increase in frequency and duration of disease symptom-free periods, or a prevention of impairment or disability due to the disease affliction.

A conjugate or composition of the present invention may be administered via one or more routes of administration, using one or more of a variety of methods known in the art. As will be appreciated by the skilled worker, the route and/or mode of administration will vary depending upon the desired results. Routes of administration for conjugates of the invention include, e.g., intravenous, intramuscular, intradermal, intraperitoneal, subcutaneous, spinal or other parenteral routes of administration, for example by injection or infusion. The phrase “parenteral administration” as used herein refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

In other embodiments, a conjugate or composition of the invention may be administered by a non-parenteral route, such as a topical, epidermal or mucosal route of administration, for example, intranasally, orally, vaginally, rectally, sublingually or topically.

As described elsewhere herein, an active conjugate may be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compounds or compositions of the invention may be administered with one or more of a variety of medical devices known in the art. For example, in one embodiment, a therapeutic conjugate of the invention may be administered with a needleless hypodermic injection device. Examples of well-known implants and modules useful in the present invention are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

In certain embodiments, the conjugate or composition of the invention may be formulated to ensure a desired distribution in vivo. For example, the blood-brain barrier (BBB) excludes many highly hydrophilic compounds. To target a therapeutic compound or composition of the invention to a particular in vivo location, they can be formulated, for example, in liposomes which may comprise one or more moieties that are selectively transported into specific cells or organs, thus enhancing targeted drug delivery. Exemplary targeting moieties include folate or biotin; mannosides; antibodies; surfactant protein A receptor; p120 and the like.

### Kits and Delivery Devices

Also, within the scope of the invention are kits comprising at least one conjugate of the invention, and optionally, instructions for use in accordance with the methods of the invention. Kits may be useful for quantifying amounts of the conjugates in a sample, or may be useful for detection of the conjugate. The kit may further or alternatively comprise at least one nucleic acid encoding a conjugate of the invention. A kit of the invention may optionally comprise at least one additional reagent (e.g., diluents, standards, markers and the like). Kits typically include a label indicating the intended use of the contents of the kit.

In certain embodiments, the invention relates to a device comprising one or more conjugates of the invention, or pharmaceutically acceptable salts or solvates thereof, for delivery to a subject. Thus, one or more compounds of the invention or pharmaceutically acceptable salts or solvates thereof can be administered to a patient in accordance with the present invention via a variety of delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety.

### EXAMPLES

The examples presented herein represent certain embodiments of the present invention. However, it is to be understood that these examples are for illustration purposes only and do not intend, nor should any be construed, to be wholly definitive as to conditions and scope of this invention. The examples were carried out using standard techniques, which are well known and routine to those of skill in the art, except where otherwise described in detail.

### Example 1. Construction and Purification of TfR-binding VNAR Conjugates with TrkB-AAbs

Construct Design. A mouse-human chimeric TrkB-AAb was prepared by inserting the antigen binding regions of mouse TrkB 29D7 Mab into a human IgG1 backbone to generate a humanized TrkB-AAb. To construct the chimeric antibody, mouse hybridoma cell line (PTA-6949), which expresses the 29D7 Mab, was obtained from the ATCC and its antibody genes were sequenced. The VH and VL domains of 29D7 were then genetically fused to the constant regions of the human kappa light chain and IgG1, respectively. Additionally, the Fc domain was modified to contain a LALA mutation to attenuate effector function of the antibody.

Two conjugates were constructed by genetically fusing the BBB shuttle TXB4 VNAR to different sites on the chimeric TrkB-AAb. The TrkB(HC2N) conjugate was prepared by attaching the TXB4 to the N-terminal end of the TrkB antibody heavy chain via a GlyGlyGlyGlySerGlyGlyGlyGlySerGlyGlyGlyGlySer linkage (3×G4S linker; SEQ ID NO. 8). The TrkB(HV2N) conjugate was prepared by inserting TXB4 VNAR between the CH1 and Fc domains of the TrkB heavy chain via a 3×G4S linker on the N-terminal end and 1×G4S linker on C-terminal end of TXB4. A control conjugate was constructed with TXB4 fused to the N-terminal end of the Fc domain (VNAR-Fc). The various conjugates are depicted in FIG. 1 while the sequences of the light and heavy chains for TrkB 29D7 and the various constructs are provided in Table 1.

TABLE 1
TXB4-TrkB Antibody Conjugate

| SEQ | |
|---|---|
| ID NO. | Antibody Chain and Amino Acid Sequence |
| | TrkB 29D7 light chain |
| 9 | AIVLIQSPATLSVTPGDSVSLSCRASQTISNNLHWYQQKSHES |
| | PRLLIKSASLAISGIPSRFSGSGSGTDFTLSISSVETEDFGMY |
| | FCQQSNSWPNTFGGGTKLEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | TrkB 29D7 heavy chain |
| 10 | EVQLQQSGPELVKPGASMKISCKTSGYSFTAYFMNWVKQSHGK |
| | SLEWIGRINPNNGDTFYTQKFKGKATLTVDKSSNTAHMELLSL |
| | TSEDSAIYYCGRRDYFGAMDYWGQGTSVTVSSASTKGPSVFPL |
| | APSSKSTSGGTAALGCLVKDYFPEPVTVSWNSGALTSGVHTFP |
| | AVLQSSGLYSLSSVVTVPSSSLGTQTYICNVNHKPSNTKVDKK |
| | VEPKSCDKTHTCPPCPAPEAAGGPSVFLFPPKPKDTLMISRTP |
| | EVTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQ |
| | PENNYKTTPPVLDSDGSFFLYSKLTVDKSRWQQGNVFSCSVMH |
| | EALHNHYTQKSLSLSPGK |
| | TrkB(HC2N) light chain |
| 9 | AIVLIQSPATLSVTPGDSVSLSCRASQTISNNLHWYQQKSHES |
| | PRLLIKSASLAISGIPSRFSGSGSGTDFTLSISSVETEDFGMY |
| | FCQQSNSWPNTFGGGTKLEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | TrkB(HC2N) heavy chain |
| 11 | ARVDQTPQTITKETGESLTINCVLRDSNCALSSTYWYRKKSGS |
| | TNEENISKGGRYVETVNSGSKSFSLRINDLTVEDSGTYRCNVV |
| | QYPQYPNYFWCDVYGDGTAVTVNAGGGGSGGGGSGGGGSEVQL |
| | QQSGPELVKPGASMKISCKTSGYSFTAYFMNWVKQSHGKSLEW |
| | IGRINPNNGDTFYTQKFKGKATLTVDKSSNTAHMELLSLTSED |
| | SAIYYCGRRDYFGAMDYWGQGTSVTVSSASTKGPSVFPLAPSS |
| | KSTSGGTAALGCLVKDYFPEPVTVSWNSGALTSGVHTFPAVLQ |
| | SSGLYSLSSVVTVPSSSLGTQTYICNVNHKPSNTKVDKKVEPK |
| | SCDKTHTCPPCPAPEAAGGPSVFLFPPKPKDTLMISRTPEVTC |
| | VVVDVSHEDPEVKFNWYVDGVEVHNAKTKPREEQYNSTYRVVS |
| | VLTVLHQDWLNGKEYKCKVSNKALPAPIEKTISKAKGQPREPQ |
| | VYTLPPSRDELTKNQVSLTCLVKGFYPSDIAVEWESNGQPENN |
| | YKTTPPVLDSDGSFFLYSKLTVDKSRWQQGNVFSCSVMHEALH |
| | NHYTQKSLSLSPGK |
| | TrkB(HV2N) light chain |
| 9 | AIVLIQSPATLSVTPGDSVSLSCRASQTISNNLHWYQQKSHES |
| | PRLLIKSASLAISGIPSRFSGSGSGTDFTLSISSVETEDFGMY |
| | FCQQSNSWPNTFGGGTKLEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | TrkB(HV2N) heavy chain |
| 12 | EVQLQQSGPELVKPGASMKISCKTSGYSFTAYFMNWVKQSHGK |
| | SLEWIGRINPNNGDTFYTQKFKGKATLTVDKSSNTAHMELLSL |
| | TSEDSAIYYCGRRDYFGAMDYWGQGTSVTVSSASTKGPSVFPL |
| | APSSKSTSGGTAALGCLVKDYFPEPVTVSWNSGALTSGVHTFP |
| | AVLQSSGLYSLSSVVTVPSSSLGTQTYICNVNHKPSNTKVDKK |
| | VEPKSCGGGGSGGGGSGGGGSARVDQTPQTITKETGESLTINC |
| | VLRDSNCALSSTYWYRKKSGSTNEENISKGGRYVETVNSGSKS |
| | FSLRINDLTVEDSGTYRCNVVQYPQYPNYFWCDVYGDGTAVTV |
| | NAGGGGSDKTHTCPPCPAPEAAGGPSVFLFPPKPKDTLMISRT |
| | PEVTCVVVDVSHEDPEVKFNWYVDGVEVHNAKTKPREEQYNST |
| | YRVVSVLTVLHQDWLNGKEYKCKVSNKALPAPIEKTISKAKGQ |
| | PREPQVYTLPPSRDELTKNQVSLTCLVKGFYPSDIAVEWESNG |
| | QPENNYKTTPPVLDSDGSFFLYSKLTVDKSRWQQGNVFSCSVM |
| | HEALHNHYTQKSLSLSPGK |

Expression and Purification. The DNA for heavy and light chain for each antibody or conjugate was synthesized as described above and cloned into its own plasmid, pEvi3 (Evitria AG, Switzerland) under the control of a mammalian promoter and polyadenylation signal. Plasmid DNA was amplified in Escherichia coli and DNA was purified using anion exchange kits for low endotoxin plasmid DNA preparation. The plasmid DNA pairs were co-transfected into CHO K1 cells with eviFect (Evitria), and the cells cultured in eviMake (Evitria), a serum-free, animal-component-free medium. Production was terminated once viability reached 75%, which occurred at day 8 after transfection.

To purify the antibody or conjugate, the CHO cell supernatant was spun at 8000×g for 20 min and filter sterilized by passing through a 0.22 um filter. Purification was done at room temperature (RT) by Protein A affinity chromatography on a AKTA Xpress instrument with subsequent size exclusion chromatography (SEC) using Superdex200 and followed by buffer exchange to phosphate-buffered saline (PBS). Samples were sterile-filtered, aliquoted and frozen at −80° C. Purity of the antibodies and conjugates was confirmed by analytical SEC as well as SDS-PAGE.

### Example 2. VNAR-Mediated In Vivo Transport of TXB4-TrkB-AAb Conjugates Across the Blood Brain Barrier

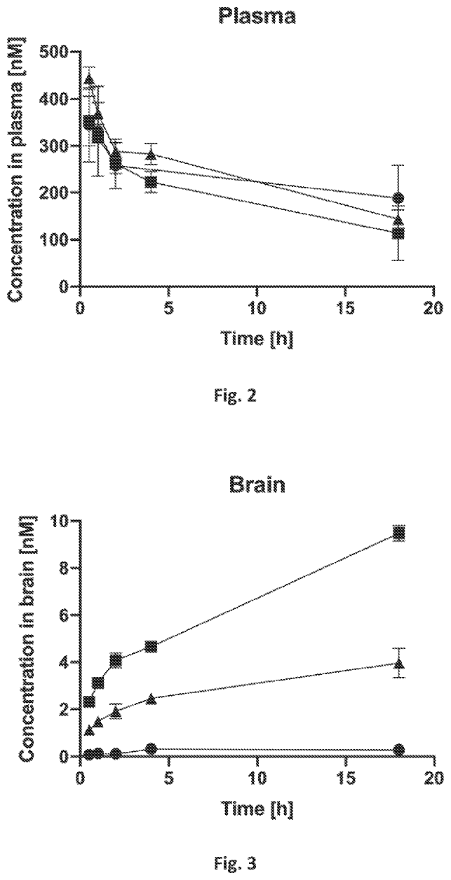

To evaluate the pharmacokinetics of TXB4-TrkB-AAb in plasma and brain, mice tail veins were intravenously injected with the conjugates and controls described in Example 1 at a dose of 25 nmol/kg. The plasma samples and brains were collected at 0.5, 1, 2, 4 and 18 hours post injection. The TfR-1-binding ELISA was used to evaluate plasma concentration in the samples, which showed half-life range for unconjugated TrkB, TrkB(HC2N) and TrkB(HV2N) of 22.8-26.6, 13.7-21.3 and 14.8-15.3 hours, respectively (FIG. 2).

Whole brain homogenates were analyzed using a brain uptake ELISA format described below. The results showed that on average TrkB(HC2N) and TrkB(HV2N) had 25.6 and 11.7 fold higher brain exposure, respectively, than the unconjugated TrkB antibody (FIG. 3). The concentration at the 18 hour time point was 0.29 nM for unconjugated TrkB, 9.48 nM for TrkB(HC2N) and 3.96 nM for TrkB(HV2N) (FIG. 3).

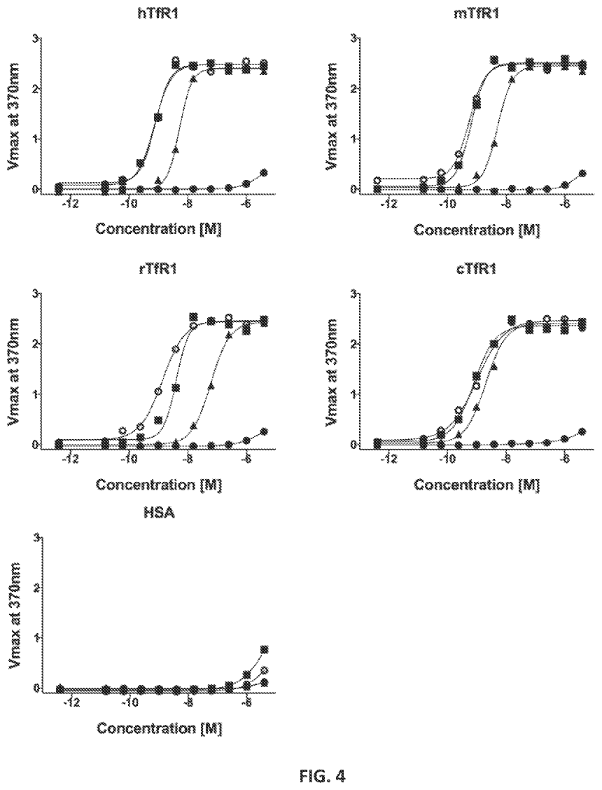

The TrkB fusion constructs were assessed for binding to recombinant human, mouse, rat and cynomolgus TfR1. TrkB(HC2N) and TrkB(HV2N) showed cross-species reactivity to all tested TfR1s (FIG. 4, Table 2). When these conjugates were compared to a control VNAR-Fc (open circles), no loss of affinity for TrkB(HC2N) (squares) was observed and only minimal loss for TrkB(HV2N) (triangles). TrkB(HC2N) and the VNAR-Fc showed sub nM affinity for all of the tested TfR1s except rat TfR-1 that produced single digit nM affinity. For TrkB(HV2N), affinity was in the single digits nM for all TfR-1 except for rat TfR-1 which exhibited an affinity of 61 nM (FIG. 4, Table 2). Unconjugated TrkB-AAb showed no binding to TfR-1. None of the conjugates or controls exhibited unspecific binding to HAS, a negative control (FIG. 4, Table 2).

TfR-1-binding ELISA. Nunc MaxiSorp plates (Thermo Fisher) were coated with 100 μl of 1 μg/ml of purified, recombinant mouse, human, rat or cynomolgus TfR-1, or with human serum albumin (HAS; Sigma) and incubated at 4° C. overnight. Plates were incubated with blocking buffer (2.5% non-fat dry milk in PBS with 0.1% Tween20; “PBST”) for 1 hat room temperature. Antibody or conjugate solutions were incubated in PBST for 30 min. The blocked solutions (100 μl) were transferred to the blocked plates and incubated for 1 h before washing with PBST. After washing, the plates were incubated for 30 min with a goat anti-human Fc-peroxidase antibody diluted 1:5000 (Sigma) in PBST. The plates were washed and developed with SureBlue™ TMB substrate (VWR). The development of the reaction was measured at 370 nm in real time providing data for Vmax analysis. EC50 values were calculated using 4-parametric non-linear regression (Prism).

Brain uptake ELISA. MaxiSorp plates were coated with 100 μl of goat anti-human Fc antibody (Sigma) diluted 1:500 in PBS or anti-VNAR antibody overnight at 4° C. The plates were washed and incubated with PBST for 1 hour at room temperature. Brains were homogenized in 3:1 (v/w) of PBST supplemented with protease inhibitors (cOmplete™, Sigma) using the TissueRuptor (Qiagen) at medium speed for 10 sec and incubated for 30 min on ice. Lysates were spun down at 17,000×g for 20 min, the supernatant was collected and blocked in 2.5% milk in PBST overnight at 4° C. Blocked brain lysates (100 μl) were added to the blocked plates and incubated for 1 hour at room temperature. The plates were washed with PBST and incubated with a goat anti-human Fc—peroxidase antibody diluted 1:5000 (Sigma) in blocking buffer for 30 min. The plates were washed and developed with SureBlue™ TMB substrate (VWR), the reaction stopped with 1% HCl and absorbance measured at 450 nm. The VNAR-Fc concentration was determined from standard curves prepared individually for each conjugate or control protein.

TABLE 2

| EC50 [nM] | hTfR1 | mTfR1 | rTfR1 | cTfR1 | HSA |
|---|---|---|---|---|---|
| TrkB | ND | ND | ND | ND | ND |
| TrkB(HC2N) | 0.74 | 0.63 | 4.03 | 0.79 | ND |
| TrkB(HV2N) | 5.3 | 5.07 | 61 | 2.04 | ND |
| VNAR-Fc | 0.78 | 0.54 | 1.37 | 1 | ND |

ND: not detected

### Example 3. TrkB Agonist Activity of the TXB4-TrkB-AAb Conjugates

The activities of unconjugated TrkB antibody and the two TXB4-TrkB-AAb conjugates were measured using TrkB-NFAT-bla CHO-K1 reporter cells that overexpress human TrkB receptor. The activity levels were compared to BDNF as positive control.

Briefly, TrkB-NFAT-bla CHO-K1 reporter cells (Invitrogen) were passaged twice a week in growth media consisting of DMEM-GlutaMAX medium (Gibco) supplemented with 10% dialysed fetal bovine serum, 100 U/mL penicillin, 100 μg/ml streptomycin, 5 μg/ml blasticidin, 200 μg/ml zeocin, 0.1 mM non-essential amino acid solution (NEAA), and 25 mM HEPES buffer (Sigma). For the assay, 2×10⁴ cells were seeded in each well of a black-wall clear-bottom 96-well plate (Corning) in 100 μl of assay media consisting of DMEM-GlutaMax medium supplemented with 0.5% dialysed fetal bovine serum, 100 U/ml penicillin, 100 μg/ml streptomycin, 0.1 mM NEAA and 25 mM HEPES. Cells were incubated overnight at 37° C., 5% CO₂. BDNF (Peprotech), unconjugated TrkB antibody and the two TXB4-TrkB-AAb conjugates were prepared in assay media and 50 μl added per well to achieve the indicated final concentration range. Cells were incubated with the BDNF and the proteins for 4 hours at 37° C., 5% CO₂ follow by assaying according to the manufacturer's recommended protocol. For the assay, 30 μl membrane-permeant fluorescent substrate coumarin cephalosporin fluorescein-acetoxymethyl ester (CCF4-AM and CCF2-AM, ThermoFisher) was added per well and incubated at room temperature for 90 minutes while protected from light. Since β-lactamase cleaves CCF2 substrate causing a shift in FRET emission, the excitation filter was set at 405 nm, and the emission filters at 460 and 530 nm on the plate reader (FlexStation, Molecular Devices). The ratio of the two emission wavelengths (λ1/λ2) was calculated as a measure of β-lactamase activity, which in turn is an indicator of TrkB receptor activation.

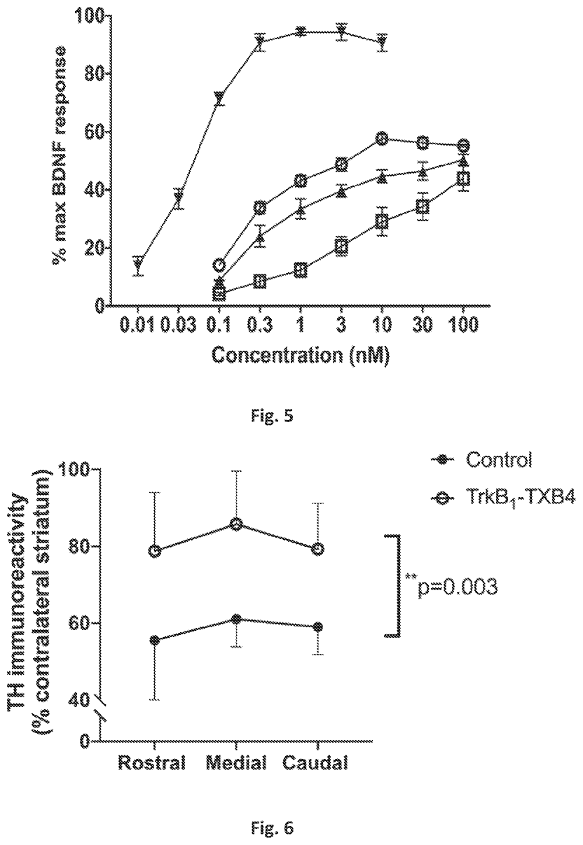

The results are shown in FIG. 5. BDNF (inverted triangles) stimulated a robust response with an EC50 determined as ˜0.055 nM (3 independent experiments). Dose response curves for the unconjugated TrkB antibody and the two TXB4 fusion constructs were presented as a percentage of the response to a maximally active concentration of BDNF. Unconjugated TrkB antibody, Trkb(HC2N) and TrkB(HV2N) reached ˜50% of the BDNF response, confirming the partial agonism previously observed with the 29D7 TrkB antibody. The activity of the unconjugated TrkB antibody and TrkB(HV2N) was similar with significant response seen at 0.3 nM and with both reaching the same maximal response at ˜10 nM. In contrast the activity of TrkB(HC2N) was estimated at ˜7-fold less than TrkB(HV2N).

### Example 4. TXB4-TrkB-AAb Conjugate Activity in a Parkinson's Disease Model

Mice with brain lesions induced by the neurotoxin 6-hydroxydopamine (6-OHDA) provide a model of neurodegeneration, and particularly a model for Parkinson's disease, for assessing the neuroprotective potential of therapeutics (see, e.g., Simola 2007). The conjugate TrkB(HV2N) was evaluated in this model and found to be neuroprotective.

6-OHDA unilateral lesion model of Parkinson's disease. A single subcutaneous (s.c.) injection of either PBS (N=5), 5 mg/kg of unconjugated TrkB antibody (N=4) or TrkB(HV2N) (N=4) (at molar equivalent) was given to male BalbC mice 24 hours prior to surgery and a second PBD injection of dose of the above proteins at 2.5 mg/kg administered at day 7.

For 6-OHDA lesioning, following anaesthesia induction (5% isofluorane/oxygen), animals were placed in a stereotaxic frame with blunt ear bars. Anaesthesia was maintained at 3% isofluorane/oxygen and body temperature maintained at 37° C. The surgical site was sterilised with 0.4% chlorhexidine (Hibiscrub) before making an antero-proximal incision along the scalp. Fine-bore holes (Ø 0.5 mm) were made in the skull at coordinates AP: +0.5 mm and ML: +2.2 mm (relative to bregma and skull surface) through which a blunt-ended 30-gauge needle was inserted to DV: −3.5 mm. 6-OHDA.HBr (4 μg in 3 μl 0.02% ascorbate/saline) was infused unilaterally into the striatum (0.5 μl/min) and the needle withdrawn 5 min later. After suturing, animals received a single dose of buprenorphine (Vetergesic; 0.1 mg/kg; s.c.) for analgesia and 1 ml of rehydrating Hartmann's solution was administered s.c. daily for 5 days. One animal in the unconjugated TrkB antibody group failed to recover adequately from surgery and was therefore excluded from the study.

Immunohistochemical assessment of TH+ cell bodies in the SNc and TH+ nerve fibres in the striatum. On post-lesion day 14, animals were euthanized by overdose of phenobarbital (Euthatal; 200 mg/ml, 1 ml injection) before being intracardially perfused with phosphate-buffered saline followed by 10% neutral buffered formalin (Sigma). Brains were removed and submerged in 10% neutral buffered formalin for 24 hours before embedding in paraffin wax. Serial 7 μm coronal sections encompassing the rostral, medial and caudal striatum and substantia nigra pars compacta (SNc) were obtained and processed for tyrosine hydroxylase (TH) staining.

Sections were dewaxed (2×5 min in xylene, 4×2 min 100% IMS) and endogenous peroxidases quenched by immersion in 3% H₂O₂ for 10 min. Antigen retrieval was performed by boiling sections in 1 mM citric acid pH 6.0 for 10 min. Blocking solution (1% bovine serum albumin (BSA) in 0.05 M Tris-buffered saline (TBS), pH7.6) was applied for 10 min before sections were incubated at room temperature overnight in a humidified chamber with primary polyclonal rabbit anti-TH antibody at 1:500 (ab152, Millipore). Primary antibody was washed off sections for 5 min in TBS and then incubated with biotinylated goat anti-rabbit secondary antibody at 1:500 at room temperature for 1 hour (BA-1000, VectorLabs). The secondary antibody was washed off sections for 5 min in TBS followed by incubation with streptavidin-biotinylated horseradish peroxidase conjugate (Vectastain Elite ABC Kit, PK6100, VectorLabs) for 30 min at RT. Color was developed using 3-3′-diaminobenzidine (DAB) substrate kit (DAB Peroxidase HRP Substrate Kit, SK-4100, VectorLabs). Finally, sections were rinsed thoroughly in distilled H₂O for 10 min to remove any trace of DAB and then dehydrated in 100% IMS (4×2 minutes) and cleared in xylene (2×5 min). Slides were mounted with coverslips using the solvent based plastic mountant DPX (Sigma).

Photomicrographs of TH-immunostained SNc sections (N=3-6 per mouse at each of the caudal [AP: −3.52 mm], medial [AP: −3.16 mm] and rostral [AP: −2.92 mm] levels relative to bregma) were acquired at 10× magnification using a Zeiss fluorescent microscope and Axiovision software (Carl Zeiss Ltd). ImageJ software was used to manually count viable TH-positive A9 dopaminergic cells of the SNc in both the lesioned and intact hemispheres (i.e., intact round cells with a clear nucleus and cytoplasm). SNc cell number in the lesion hemisphere was calculated as percentage of that remaining in the intact hemisphere. Data were combined across all three rostro-caudal levels to generate a single average value for each animal. Mean data were then calculated per treatment group.

Photomicrographs of TH-immunostained striatal sections (N=3-6 per mouse at each of the caudal [AP: −0.22 mm], medial [AP: +0.5 mm] and rostral [AP: +1.0 mm] levels relative to bregma) were acquired at 10× magnification using a Zeiss fluorescent microscope and Axiovision software (Carl Zeiss Ltd). ImageJ software was used for densitometric analysis using the mean grey value function and corrected for background cortical stain. TH intensity in the lesioned hemisphere was determined as a percentage of the contralateral hemisphere for each animal at the rostral, medial and caudal levels. Mean data at each level for calculated for individual animals and the average of this value used to calculate the value for individual treatment groups.

Serial 7 μm sections of the SN were obtained and processed for TH staining as described above. Photomicrographs of TH-immunostained SNc sections (n=3 per mouse at each of the caudal [AP: −6.0 mm], medial [AP: −5.3 mm] and rostral [AP: −4.8 mm] levels) were acquired at 20× magnification using a Zeiss fluorescent microscope and Axiovision software (Carl Zeiss Ltd). ImageJ software was used to manually count viable TH-positive A9 dopaminergic cells of the SNc in both the lesioned and contralateral hemispheres. SNc cell number in the lesion hemisphere was calculated a percentage of that in the contralateral hemisphere. Mean data at each level calculated for individual animals and the average thereof used to calculate the value for individual treatment groups.

This study was performed in accordance with UK Animals Scientific Procedures Act (1986) and was approved by King's College London Animal Welfare and Ethical Review Body. All animals were maintained on a 12:12 hour light:dark cycle with food and water available ad libitum.

Results. As described above, a dose of 6-OHDA predicted to allow a partial lesion to develop over a 2-week period was injected into the left striatum on day 0. Experimental animals (N=x) received a subcutaneous injection of 5 mg/kg TrkB(HV2N) on day −1 and of 2.5 mg/kg on day 7. Control animals (N=x) received a subcutaneous injection of PBS on day −1 and on day 7. Brains were isolated on day 14 and processed for TH immunoreactivity in the rostral, medial and caudal striatum. The immunoreactivity on the lesioned side of the brain was ratioed against that of the contralateral brain in the same section.

TH immunoreactivity was reduced ˜40% throughout the striatum in the PBS-treated control group (FIG. 6). This was statistically significant (p<0.05) for each brain area relative to the respective contralateral side of the brain in the same animal group. In contrast, there was a variable but clearly muted response in the TrkB(HV2N) treatment group with the reductions in individual areas failing to reach statistical significance relative the contralateral side of the brain in the same animal group. Furthermore, when consideration was given to the magnitude of the lesion across the rostral-caudal axis in the striatum, lesioning was clearly reduced (p=0.003) in the TrkB(HV2N) group relative to the PBS control group (FIG. 6).

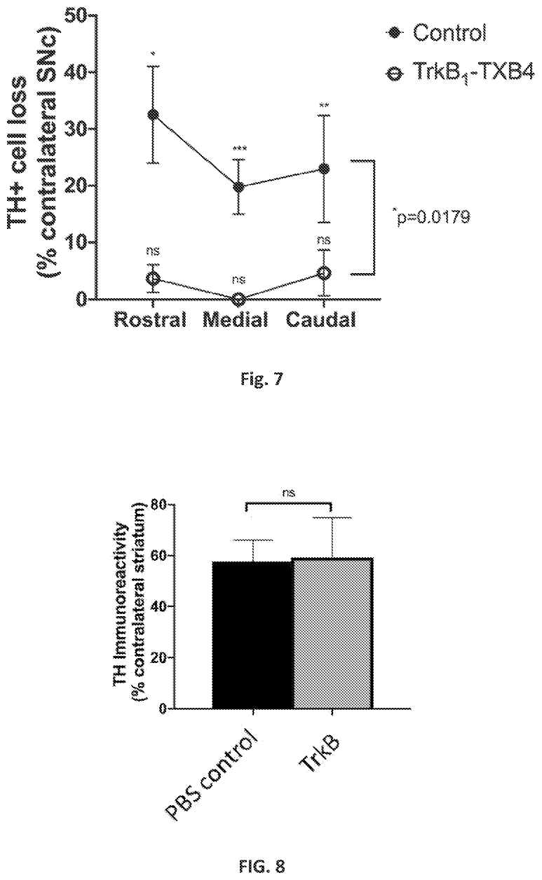

The number of TH positive neurons in rostral, medial and caudal regions of the SNc in the same animal groups was counted. Reductions on the lesioned side of the brain relative to the contralateral side ranged from 32.5+/−12 (P<0.05) in the rostral region, 19.8+/−4.8 (P<0.0001) in the medial region and 23.0+/−9. (P<0.001) in the caudal region (all values mean±SEM) (FIG. 7). In stark contrast, no significant loss of any neurons occurred in any region of the SNc in the TrkB(HV2N) treatment group relative to the contralateral brain in the same group (FIG. 7). Again, when consideration was given to the magnitude of neuronal loss across the rostral-caudal axis in the SNc, neuronal loss was clearly reduced (p=0.0179) in the TrkB(HV2N) group relative to the PBS control group (FIG. 7).

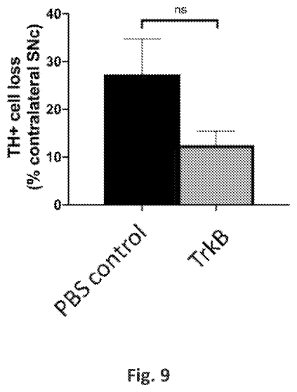

Comparisons were made between the 6-OHDA lesions seen in animals treated with the unconjugated TrkB antibody relative to the PBS control. The lesions in the striatum of both animal groups measured throughout the striatum were the same with approximately 40% reduction in TH immunoreactivity (FIG. 8). TH-positive cell numbers in the SNc showed a non-significant (p=0.249) trend towards a protective effect of the unconjugated TrkB antibody (FIG. 9).

### REFERENCES

Bai et al. (2010) An agonistic TrkB mAb causes sustained TrkB activation, delays RGC death, and protects the retinal structure in optic nerve axotomy and in glaucoma. Invest Ophthalmol Vis Sci. 51(9):4722-31.

Cazorla et al. (2011) Pharmacological characterization of six trkB antibodies reveals a novel class of functional agents for the study of the BDNF receptor. Br J Pharmacol. 162(4):947-60.

Fouad et al. (2010) A TrkB antibody agonist promotes plasticity following cervical spinal cord injury in adult rats. J Neurotrauma. 2010 Jul. 2.

Greenberg et al. (1995) A new antigen receptor gene family that undergoes rearrangement and extensive somatic diversification in sharks. Nature 374(6518):168-73).

Han et al. (2019) Therapeutic potential of a TrkB agonistic antibody for ischemic brain injury. Neurobiol Dis. 2019 July; 127:570-581. Epub 2019 Apr. 11.

Hu et al. (2010) Neurotrophic effect of a novel TrkB agonist on retinal ganglion cells. Invest Ophthalmol Vis Sci. 51(3):1747-54.

Johnsen et al. (2019) Targeting the transferrin receptor for brain drug delivery. Prog. Neurobiol. 181:101665.

Josephy-Hernandez S. et al., Neurobiology of Disease 97 (2017) 139-155; available online 18 Aug. 2016 at Elsevier;

Kim et al. (2014) TrkB agonist antibody pretreatment enhances neuronal survival and long-term sensory motor function following hypoxic ischemic injury in neonatal rats. PLoS One. 9(2):e88962.

Merkouris et al. (2018) Fully human agonist antibodies to TrkB using autocrine cell-based selection from a combinatorial antibody library. Proc Natl Acad Sci U S A. 115(30):E7023-E7032. Epub 2018 Jul. 9.

Perreault et al. (2013) Activation of TrkB with TAM-163 results in opposite effects on body weight in rodents and non-human primates. PLoS One. 8(5):e62616.

Qian et al. (2006) Novel agonist monoclonal antibodies activate TrkB receptors and demonstrate potent neurotrophic activities. J. Neurosci. 26(37):9394-403.

Rosenthal et al. (2014) Modulation of neurotrophin signaling by monoclonal antibodies. Handb Exp Pharmacol. 2014;220:497-512.

Sahenk et al. (2010) TrkB and TrkC agonist antibodies improve function, electrophysiologic and pathologic features in Trembler J mice. Exp Neurol. 224(2):495-506.

Simola et al. (2007) The 6-hydroxydopamine model of Parkinson's disease. Neurotox Res. 11(3-4):151-67.

Szobota et al. (2019) BDNF, NT-3 and Trk receptor agonist monoclonal antibodies promote neuron survival, neurite extension, and synapse restoration in rat cochlea ex vivo models relevant for hidden hearing loss. PLoS One. 14(10):e0224022.

Todd et al. (2014) A monoclonal antibody TrkB receptor agonist as a potential therapeutic for Huntington's disease. PLoS One. 9(2):e87923.

Vugmeyster et al. (2013) Agonistic TAM-163 antibody targeting tyrosine kinase receptor-B: applying mechanistic modeling to enable preclinical to clinical translation and guide clinical trial design. MAbs. 5(3):373-83.

Xu et al. (2010) TrkB agonist antibody dose-dependently raises blood pressure in mice with diet-induced obesity. Am J Hypertens. 23(7):732-6.

1. A conjugate comprising at least one BBB-shuttling VNAR domain operably linked to a neurotrophic agonist antibody (NAAb), said conjugate being capable of uptake across a mammalian blood brain barrier (BBB) in a therapeutically-effective amount, wherein said neurotrophic agonist antibody is a TrkA, TrkB or TrkC agonist antibody or an antigen binding fragment thereof.

2. The conjugate of claim 1 wherein said BBB-shuttling VNAR domain is
(a) a TfR-binding VNAR domain capable of specifically binding to a human TfR-1 without substantially interfering with transferrin binding to and/or transport by said human TfR-1,
(b) a TfR-binding VNAR domain capable of specifically binding to a human TfR-1 without substantially interfering with transferrin binding to and/or transport by said human TfR-1 and capable of cross reacting with mouse TfR-1, or
(c) a TfR-binding VNAR domain capable of binding human TfR-1 with an EC50 ranging from about 1 nM to about 800 nM.

3. The conjugate of claim 1 wherein said BBB-shuttling VNAR domain is
(a) a TfR-binding VNAR domain designated as Clone C or one of its variants,
(b) a TfR-binding VNAR domain designated as Clone H or one of its variants,
(c) the a TfR-binding VNAR domain designated as Clone 8 or one of its variants
(d) TXB4,
(e) a CD98-binding VNAR domain, or
(f) VNAR-txp1 domain.

4. (canceled)

5. The conjugate of claim 1, wherein said neurotrophic agonist antibody is a TrkB agonist antibody or an antigen binding fragment thereof.

6. The conjugate of claim 5, wherein said TrkB agonist antibody is monoclonal antibody 29D7, is a chimeric, humanized or veneered version of monoclonal antibody 29D7 or is an antigen-binding fragment of any of the foregoing.

7. (canceled)

8. The conjugate of claim 1 comprising two or more independent BBB-shuttling VNAR domains.

9. (canceled)

10. The conjugate of claim 8, wherein said conjugate is TrkB(HC2N) or TrkB(HV2N).

11. The conjugate of claim 1 which comprises a diagnostic agent or a further therapeutic agent.

12.-13. (canceled)

14. A pharmaceutical composition comprising a conjugate of claim 1.

15. A method of treating a neurodegenerative disease or a condition which responds to activation of a neurotrophin receptor which comprises administering a therapeutically-effective amount of the pharmaceutical composition of claim 14 to a mammalian subject in need thereof for a time and in an amount effective to treat said disease or condition.

16. The method of claim 15, wherein said conjugate is administered intravenously, intramuscularly, subcutaneously, intraarterially, intracranially or intrathecally, preferably, intravenously.

17. The method of claim 15, wherein administering said conjugate causes delivery thereof to the brain of said mammalian subject to thereby treat said disease or condition.

18. The method of claim 16, wherein said neurodegenerative disease or condition is Parkinson's disease, an acute or chronic neurological injury or wound, amyotrophic lateral sclerosis (ALS), or Alzheimer's disease (AD).

19. (canceled)

20. The method of claim 15, wherein said conjugate comprises two BBB-shuttling VNAR domains that bind human TfR-1 operably linked to a TrkB agonist antibody or an antigen binding fragment thereof.

21. The method of claim 20, wherein said conjugate is TXB4 operably linked to a TrkB agonist antibody.

22. The method of claim 21, wherein said conjugate is TrkB(HC2N) or TrkB (HV2N.

23. (canceled)

24. A method of stimulating neuronal survival, growth, repair or regeneration which comprises contacting a population of neurons with a conjugate of claim 1.

25.-26. (canceled)

27. A nucleic acid encoding the conjugate of claim 1.

28. A vector comprising a nucleic acid of claim 27.

29. A host cell comprising the vector of claim 28.
